(19) United States
(12) Patent Application Publication
(10) Pub. No.: US 2003/0049320 A1
(43) Pub. Date: Mar. 13, 2003

(54) Novel in-situ forming controlled release microcarrier delivery system

(75) Inventors: Harshal P. Bhagwatwar, Aurangabad (IN); Varada R. Bapat, Aurangabad (IN); Mahesh B. Paithankar, Aurangabad (IN); Bhushan S. Yeola, Aurangabad (IN); Arun S. Gosavi, Aurangabad (IN); Manoj A. Bagool, Aurangabad (IN); Nitin Shetty, Aurangabad (IN); Milind C. Shukla, Aurangabad (IN); Noel J. De Souza, Aurangabad (IN); Nishith C. Chaturvedi, Aurangabad (IN); Suresh Beri, Aurangabad (IN)

Correspondence Address: LADAS & PARRY, 26 WEST 61ST STREET, NEW YORK, NY, 10023, US

(73) Assignee: WOCKHARDT LIMITED, MUMBAI (IN)

(21) Appl. No.: 10/023,427

(22) Filed: Dec. 12, 2001

Related U.S. Application Data
(60) Provisional application No. 60/256,319, filed on Dec. 18, 2000.

(51) Int. Cl. A61K 9/14

(52) U.S. Cl. 424/486; 424/488; 424/487

(57) ABSTRACT

A ready-to use, stable, gelled polymer droplet-in-oil dispersion is described which helps in in-situ formation of a multitude of small solid, semisolid, or gelled microcarriers. The dispersion is placed into a body in a semisolid form and cures to form the delivery system in-situ. The process for making such a dispersion comprises the steps of (i) dissolving a polymer in a biocompatible solvent at an elevated temperature to form a polymer solution, (ii) preparing a second oil phase solution of a biocompatible oil and a biocompatible emulsifier at an elevated temperature, (iii) mixing the polymer solution with the oil phase solution at an elevated temperature and subsequently cooling to refrigeration temperature. Placing the gelled dispersion within a body produces the microcarrier delivery system in-situ. The composition of a syringeable, biodegradable dispersion incorporating an effective level of a biologically active agent before injection into a body provides a novel controlled delivery system of drugs for healthcare applications.

### FIELD OF THE INVENTION

This invention relates to a novel in-situ forming controlled release microcarrier delivery system provided by a gelled composition for controlled delivery of biologically active or bioinactive materials. The gelled composition comprises a polymer, an organic solvent, an oil, and an emulsifier resulting in a ready-to-use, gelled, syringeable, solution-in-oil dispersion. This invention also relates to a process by which the composition incorporating the biologically active agent or bioinactive material is made. The use of the polymer microcarrier system and the composition for healthcare applications is also described.

### BACKGROUND OF THE INVENTION

Polymers have been used in the medical field in various forms such as sutures, surgical clips, implants, and drug delivery systems. For all of these applications, the polymers have to be processed by procedures such as for example high temperature extrusion or molding, tabletting, microencapsulation, to formulate them into their final shapes, before administration to the body. Examples of such procedures include microencapsulation procedures such as in-water drying (U.S. Pat. No. 4,652,441 to Okada et al.) for highly water-soluble drugs; solvent evaporation (U.S. Pat. No. 4,389,330 to Tice et al.) for water-insoluble drugs; method-dependent coacervation-phase separation (U.S. Pat. No. 5,603,960 to O'Hagan et al.) for water-soluble or insoluble drugs; spray drying (U.S. Pat. No. 5,622,657 to Takada et al.), solvent extraction (U.S. Pat. No. 4,389,330 to Tice et al.), polymer droplet-in-oil solvent evaporation-extraction (U.S. Pat. No. 5,705,197 to Van Hamont et al.), and extrusion processes for the formation of solid polymeric implants (U.S. Pat. No. 5,945,128 to Deghenghi), to name a few. All of these procedures require that the devices such as solid polymeric implants be formed outside the body and that they be administered to the body through surgical intervention, resulting in loss of patient compliance. In other cases, preformed microparticles have to be reconstituted first with an aqueous vehicle which also acts as a suspending agent before being administered via syringe-and-needle assemblies. In addition, these procedures suffer from several disadvantages with respect to scale-up, use and removal of residual toxic often carcinogenic volatile organic solvents, use of different techniques such as oil-in-water and water-in-oil-in-water, in-water drying techniques for drugs with different physicochemical characteristics, to name a few.

U.S. Pat. Nos. 4,938,763; 5,278,201; 5,945,115 and 5,702,716 to Dunn et al., and U.S. Pat. Nos. 5,620,700 and 5,783,205 to Berggren et al. describe injectable formulations for forming implants in-situ comprising solutions or suspensions of biologically active drug substances in a solution of a thermoplastic polymer in a biocompatible water-miscible organic solvent. These formulations assume the shape of the cavity into which they are administered to form a single, monolithic, “space-filling” implant which solidifies upon coming in contact with body fluids through the dissipation of the water-miscible organic solvent and precipitation of the polymer. The use of these formulations, however, is more for space-filling implants and generally for periodontal treatment, bone regeneration, wound treatment and the like and not for drug delivery for which they pose some major problems including variability in the rates of solidification, shapes of the implants formed depending upon the cavity into which the formulation is introduced, undesirable high initial bursts of the drug of up to 50%, injection of large amounts of solvents into the body and addition of preformed microparticles into the vehicle to control the release.

U.S. Pat. No. 4,631,188 (Stoy et al) describes a polymeric composition comprised of water insoluble, non-crosslinked polymeric compounds having a solubility parameter of between 9.2 and 15.5 (cal/cc)^{1/2} dissolved in a polar, non-toxic water miscible solvent. Stoy et al describe that the polymeric composition must be insoluble in water or blood serum.

Shimizu (Shimizu Yasumitsu; EP 1033127 A1 and WO 98/41190) describes a composition for forming microparticles in-situ comprising an emulsion of a solution of a biodegradable polymer in an organic solvent in a continuous phase comprised of a polyhydric alcohol with an added viscosity enhancer and adhesive. This composition has limited industrial applicability because the solvents used in the examples provided as the ‘Best Mode of the Invention’, namely triethyl citrate, triacetin and propylene carbonate, are water-insoluble (solubility less than 100 mg/ml water) with consequent undesirable high burst effects of 40-90% of the drug released within 24 hours and thus a proportionately low drug entrapment in the microparticles. The delivery system is designed and exemplified specifically for periodontal delivery, using biodegradable polymers only. Unlike the present invention the work by Shimizu (Shimizu Yasumitsu; EP 1033127 A1 and WO 98/41190) does not describe formation of a delivery system using non-biodegradable or water-soluble polymers and their combinations; or the formation of a delivery system for biologically active substances with a variety of physicochemical properties. Also, the controlled release of a biologically active agent over extended time periods has not been demonstrated. Additionally, propylene glycol used in the examples of the Best Mode of the Invention of Shimizu which forms the continuous phase of the composition is myotoxic (Brazeau et. al. “Mechanisms of creatinine kinase release from isolated rat skeletal muscles damaged by propylene glycol and ethanol”, J. Pharm. Sci. (1990) 79(5): 393-397). Unlike the present invention, Shimuzu, EP 1033127 A1 and WO 98/41190, does not describe the use of a continuous phase comprised of an oil stabilized by the gelling effect of sorbitan monostearate, sorbitan monopalmitate or a mixture thereof in forming in-situ polymeric microcarriers from gelled polymeric dispersions.

A multiphase system developed by Bodmeier (Bodmeier Roland, “Multiphasensystem”, WO 98/55100 A1 and EP 996426 A1, DE 19724784 A1) comprises an emulsion of a solution of a biodegradable polymer in an organic solvent in a continuous phase comprised of an oil with an added viscosity enhancer and emulsifier. This system suffers from the drawback that the dispersion has to be prepared shortly before administration (claims 19 and 67, WO 98/55100) and the two phases which are mixed to form the system have to be stored in a dual-chambered syringe in two separate compartments (claims 68-70, WO 98/55100). Several claims including the formation and the use of the composition for controlled drug delivery with reduced burst effects (claim 55, WO 98/55100), formation of the composition incorporating a variety of biologically active agents in a variety of polymers, and delivery of peptide and protein pharmaceuticals (claim 35, WO 98/55100) are not supported by any substantive data in the specification. The system requires the use of separate materials, one for viscosity enhancement and another for emulsification and is inherently unstable in the absence of a viscosity enhancer. Unlike the present invention, WO98/55100 does not describe the use of sorbitan monostearate, sorbitan monopalmitate or a mixture thereof for the formation of a ready-to-use, stable, in-situ microcarrier forming gelled polymeric dispersion without necessitating the use of an additional viscosity enhancing agent.

An in-situ microspheres forming delivery system similar to the multiphase system of Bodmeier developed by Jain et al. (“Controlled drug delivery from a novel injectable in-situ formed biodegradable PLGA microsphere system”, Ph.D. dissertation by Rajeev Jain submitted to the University of Rhode Island, USA, 1998; Jain et. al., 2000, J. Microencapsul. 17(3): 343-362; Jain et al., 2000, Pharmaceutical Development and Technology, 5(2): 201-207) has limited or no industrial applicability because of the large volumes of the formulation required to administer normal doses of potent drugs. In addition, the use of water-immiscible organic solvents, as solvents for the polymer (triacetin and triethylcitrate), poor drug loadings and high burst effects provide a formulation with limited use potential. Also, no details are provided as to preparation of the composition with molecules with a wide variety of physicochemical properties. Similarly, the formation of the delivery system is demonstrated for only two poly(dl-lactide-co-glycolide) polymers. The applicability for other classes of polymers, with different physicochemical characteristics and biodegradability profiles, is not demonstrated.

There is thus a need for a ready-to use composition for providing an in-situ forming microcarrier delivery system, using biocompatible, biodegradable or nonbiodegradable polymers, which is not space-filling and is capable of rapidly forming polymeric microcarriers delivering biologically active substances, bioinactive substances or both having a variety of physicochemical characteristics such as highly water- and solvent-soluble, but oil-insoluble, peptides/proteins and non-peptides, and water-insoluble but solvent- and oil-soluble peptides/proteins and non-peptides. The term solvent-soluble indicates that the biologically active or bioinactive substances are soluble in the water-soluble solvents used in the invention.

There is a need for a stable, syringeable composition which is capable of rapidly forming a microcarrier delivery system in-situ, allowing the administration of high doses of biologically active substances in small volumes of the composition.

There is a further need for a versatile composition which provides a method for the formation of microcarriers in-situ after administration to the body via other routes such as orally, topically, vaginally, rectally, intratumorally, intravascularly, intramuscularly, subcutaneously, intradermally, intranasally, intralesionally, buccally, ocularly, intravenously, intraperitoneally, transdermally, locally, regionally, loco-regionally, or by any other pharmaceutically acceptable route.

There is also a need for a method for large-scale production of a gelled composition that can used to form a microcarrier delivery system.

The current invention addresses several needs for a drug delivery system such as the provision of a ready-to-use, stable, gelled, polymeric dispersion, encompassing a uniformly distributed biologically active, bioinactive agent or a mixture thereof which is capable of:

(a) rapidly forming in-situ, polymeric microcarriers of a controlled size, distribution and shape upon coming in contact with an aqueous medium,

(b) efficiently entrapping biologically active, biologically inactive substances or a mixture thereof varying in physicochemical properties from highly water-soluble to highly water-insoluble, and peptidic to non-peptidic, in polymers with physico-chemical characteristics varying from biodegradable to non-biodegradable and their mixtures, with a substantially reduced burst effect of less than 30% and providing controlled release of the biologically active or biologically inactive agent over extended time periods.

The current invention also addresses the need for a delivery system for bioinactive substances.

### SUMMARY OF THE INVENTION

A novel in-situ forming microcarrier delivery system for the controlled release of biologically active agents or bioinactive agents, and a ready-to-use, stable, gelled composition for its formation is provided. The gelled composition comprises a biocompatible solid polymer or copolymer dissolved in a biocompatible water-soluble solvent (or a mixture of water-soluble solvents), to form a liquid solution, which solution is further emulsified into a continuous oil phase to form a microdroplet dispersion. On placing such a dispersion into a body where there is an aqueous component, a multitude of microcarriers is formed. In the microcarrier drug-delivery system of the invention the biologically active agent or bioinactive agent is incorporated in the polymer solution alone, or in the polymer solution as well as the continuous oil phase as a homogeneous solution or as a suspended dispersion. The release of the biologically active agent and bioinactive agent follows the general rules for release from a polymeric delivery system.

The present invention overcomes the usually encountered problems as cited earlier in the text and to be found in the prior art, namely the problems of unavailability of a ready-to-use gelled formulation, instability of the dispersions, the need to formulate just prior to administration, poor drug loadings, high volumes of formulation required for the administration of potent drugs, and large burst effects during drug release.

The present inventors have found for the first time, that certain nonionic emulsifiers such as sorbitan monostearate and sorbitan monopalmitate, which are known to gel vegetable oils (Murdan et al., 1999, J. Pharm. Sci. 88(6): 608-614), are also capable of gelling water-soluble non-volatile organic solvents such as N,N′-dimethylacetamide (DMA), dimethylsulfoxide (DMSO), N-methyl-2-pyrrolidone (NMP), 2-pyrrolidone, dimethyl formamide, caprolactam, decylmethyl sulfoxide, liquid polyethylene glycols (PEG), propylene glycol, glycerol, glycofural, glycerolformal, and sorbitol. Other water soluble non-volatile organic solvents can also be used as the solvent. Water can also be used a solvent. Thus, for example, a solution of a polymer in DMSO when emulsified into a solution of the nonionic emulsifier (sorbitan monostearate, sorbitan monopalmitate or a mixture thereof) in the oil at an elevated temperature and subsequently cooled, provides a true polymer droplet-in-oil dispersion. This dispersion is a viscous gel at temperatures of 2-8° C. but flows upon application of shear through a syringe-needle assembly. Upon coming in contact with an aqueous medium, discrete microcarriers are formed. The presence of the nonionic emulsifier of the invention in this novel dispersion allows the formation of a ready-to-use microcarrier-forming composition which causes rapid emulsification of the oil phase on contact with an aqueous medium.

In one embodiment of the invention, the physical stability of the ready-to-use gelled composition can be significantly improved at temperatures of 2-30° C. by the use of mixtures of the water-soluble solvents which are gelled by either sorbitan monostearate, sorbitan monopalmitate or both, or in conjunction with a water-soluble low-melting polymer.

It is a further embodiment of this invention that the above described gelled polymeric dispersions when dispersed in an aqueous fluid form microcarriers of a controlled particle size and shape. These gelled polymeric dispersions when loaded with a biologically active substance, provide a delivery system from which the active agent has a reproducible release profile. These gelled polymeric dispersions when loaded with a biologically inactive substance, provide a delivery system from which the bioinactive agent has a reproducible release profile.

The gelled polymeric dispersions of this invention provide advantages over the prior art methods for preparing polymer droplet-in-oil dispersions in that the compositions have advantages over the prior art, including physical stability, ready-to-use formulations with high drug loadings, capability of administration of high doses through small volumes of the formulation, rapid rates of precipitation leading to enhanced drug loadings in the in-situ formed microcarriers and low burst effects, and other advantages related to drug delivery.

The invention also includes a process for preparation of the composition of the invention which comprises the steps as detailed below.

The composition of this invention may be used in the treatment or prevention of health disorders, diseases or medical conditions. prophylatically or to treat a disease or condition.

### Advantages of the Present Invention over the Prior Art Delivery Systems

### Advantages of the Composition

1. The dispersion of the invention is ready-for-injection and no reconstitution step is involved.

2. The viscous nature of the gelled dispersion allows for exceptionally good physical stability over prolonged periods of time at temperatures of 2-30° C.

3. The use of water-soluble organic solvents obviates the use of materials such as methylene chloride, ethyl acetate, chloroform, silicone oil, and other such materials completely; thereby removing the problems of toxic carcinogenic residual solvents, atmospheric contamination and changes in product characteristics on storage.

4. The use of mixtures of solvents and polymer combinations allows a reduction in the volume of solvents to be injected into the body.

### Advantages of the Process to Make the Composition

1. The process of manufacture of the composition allows high encapsulation efficiencies of drug substances of different physicochemical characteristics such as water solubilities, partition coefficients, molecular weights, using polymers of different physicochemical and biodegradation characteristics.

2. The manufacture of the product requires a reduced number of steps thus providing high yields (85-95%) and making the product very easy to scale-up and more reproducible when compared with other existing products. The method allows for high concentration dispersions to be prepared allowing administration of high doses through small volumes of the composition.

### BRIEF DESCRIPTION OF THE FIGURES

FIG. 1 is a graph showing the controlled release of leuprolide acetate from novel polymeric dispersions prepared using different water-soluble solvents.

FIG. 2 is a graph showing the release of leuprolide acetate from gelled polymeric dispersions with different polymer combinations.

FIG. 3 is a graph showing the plasma concentration of paclitaxel following subcutaneous administration of the gelled dispersion in female Wistar rats.

FIG. 4 is a graph showing serum testosterone concentration in male Sprague Dawley rats following intramuscular administration of the gelled dispersion.

### DETAILED DESCRIPTION OF THE INVENTION

The present invention relates to a novel polymer system for the controlled delivery of biologically active or bioinactive substances, a ready-to-use, gelled, syringeable composition for producing such a system, a process for preparing and administering the composition and a method of use for such a composition and system.

The microcarrier delivery system comprises a multitude of microcarriers formed from the interaction between a gelled composition and an aqueous fluid. The gelled composition comprises a polymer, a water-soluble organic solvent, an appropriate oil which may be a vegetable or animal oil, and an emulsifier resulting in a stable, gelled, syringeable, polymer solution-in-oil dispersion, which dispersion upon administration into a body and coming in contact with aqueous fluids forms microcarriers, each of which functions as a distinct site for the controlled release of bioactive or bioinactive materials.

The novel composition of the present invention is a ready-to-use, stable, gelled polymeric dispersion formed by the mixing of a solution of a biocompatible polymer in a water-soluble organic solvent with a continuous oil phase. The composition possesses the characteristics of rapidly forming discrete semisolid to gelled microcarriers upon coming in contact with aqueous fluids. The gels are prepared in high yields (85-95%) in extremely short periods of time (in 2-3 hours compared with 3-4 days for prior art methods of microencapsulation). The gelled dispersions are ready-to-use and are stable for 8 hours-6 months, at temperatures of 2-30° C.

Another characteristic of the composition of the invention is the syringeability of the composition. The dispersion is a viscous gel at temperatures of 2-8° C. and can be easily injected via a conventional syringe-needle assembly. The syringes used could be made of glass or plastic or any other material acceptable for human or animal use. The syringes could also be prefilled syringes. The needles to be attached to the syringes could be of 10-26 gauge. The choice of the needle to be used for administration will depend upon the viscosity of the final formulation. Any needles available in the market for pharmaceutical or medicinal use are acceptable for the administration of the formulation. Of course, if the preferred route of administration is invasive such as parenterally, intratumorally, intralesionally, intraocular and such other routes, it is preferrable that the syringe-needle assembly be sterile and pyrogen free.

A further characteristic of the novel composition is that the microcarriers are formed rapidly upon coming in contact with aqueous media. The aqueous media for the purposes of the invention could be any media containing water as the principal component, containing other excipients such as buffering agents, salts, chelating agents, antioxidants, preservatives, emulsifiers, and such other excipients to be added as per the requirement of the medium. The media could be those prepared for in vitro testing or those present in a human or animal body where the formulation would be administered. Such media present in vivo could include saliva, gastrointestinal fluids, blood, serum, plasma, interstitial fluids, ocular fluids, cerebrospinal fluids, fluids accumulated in lesions and such other fluids.

The microcarriers formed from the novel polymeric dispersions are formed in high yields, generally about 60-90% and preferably at least 85%. The microcarriers are of a controlled particle size distribution with particles ranging in size from 1-400 μm, preferably 5-150 μm, with greater than 40-60% of the particles having an average particle size less than 100 μm. The shape of the microcarriers are most commonly spherical, oblong, elliptical, or irregular in shape. The size, distribution and shape of the microcarriers is controlled by the size, distribution and shape of the droplets of the polymer in the final gelled dispersion. The processing conditions such as, where applicable, the speed of homogenization, and the molecular structure of the final gel will determine the size, distribution and shape of the droplets. These characteristics are maintained by the viscous gelled nature of the dispersion. homogenization speed during manufacture.

Another important characteristic of the in-situ formed microcarriers is their semisolid to gelled consistency in contrast to the microparticles obtained by the techniques described in the prior art which are solid in consistency (U.S. Pat. No. 4,652,441 to Okada et al., U.S. Pat. No. 4,389,330 to Tice et al., U.S. Pat. No. 5,603,960 to O'Hagan et al., U.S. Pat. No. 5,622,657 to Takada et al., U.S. Pat. No. 5,705,197 to Van Hamont et al.).

The microcarriers are capable of entrapping drug substances with a variety of physicochemical characteristics such as highly water- and solvent-soluble, but oil-insoluble peptides/proteins and non-peptides, and water-insoluble but solvent- and oil-soluble peptides/proteins and non-peptides, with high loading, held mainly within the polymer droplets with very little or no drug or bioinactive agent in the continuous oil phase. Of course, certain amounts of the biologically active agent could be added to the gelled continuous oil phase to provide an initial release of the agent.

### The Biocompatible Polymer

The polymer is a long chain polymer, amorphous, semicrystalline or crystalline in nature. Preferably, the long chain polymer is one with a molecular weight in the range of 500 to 100,000 daltons as measured by gel permeation chromatography against polystyrene standards. The chosen polymer could be biodegradable or non-biodegradable. For parenteral applications, a biodegradable polymer with a degradation profile occuring within 1 week to 1 year, is desirable. Examples of such biodegradable polymers useful in this invention include but are not limited to poly-L-lactic acids, poly-DL-lactic acids, poly-L-lactides, poly-DL-lactides, poly(L-lactic acid-co-glycolic acids), poly(DL-lactic acid-co-glycolic acids), poly(L-lactide-co-glycolides), poly(DL-lactide-co-glycolide), polyglycolides, polycaprolactones, polycarbonates, polyorthoesters, polyaminoacids, polyethylene glycols, polyethylene oxides, polyvinyl alcohol, polyvinyl pyrrolidone, polyoxyethylene-polypropylene block copolymers, polyethers, polyphosphazenes, polydioxanones, polyacetals, polyhydroxybutyrates, polyhydroxyvalerates, polyhydroxycelluloses, chitin, chitosan, polyanhydrides, polyalkylene oxalates, polyurethanes, polyesteramides, polyamides, polyorthocarbonates, polyphosphoesters, star-branched polymers and copolymers, betacyclodextrin, polysaccharides, gelatin, collagen, albumin, fibrin, fibrinogen, polyketals, polyalkylene succinates, poly(malic acid), polypropylene oxides and other biodegradable polymers, known to a person skilled in the art of drug delivery and their copolymers, terpolymers, combinations and mixtures thereof.

These polymers can either be used alone or as copolymers created from the different monomers in different ratios or mixtures of two or more different polymers or copolymers to achieve a variety of release profiles and degradation rates. The copolymers could either be random copolymers in a variety of comonomer ratios or block copolymers. Such polymers could be end-blocked or free carboxylic acid endgroup polymers or mixtures of these or polymers with other end groups.

Preferred polymers are those with a lower degree of crystallinity and a higher degree of hydrophobicity. Such polymers include but are not limited to poly-L-lactic acids, poly-DL-lactic acids, poly-L-lactides, poly-DL-lactides, poly(L-lactic acid-co-glycolic acids), poly(DL-lactic-acid-co-glycolic acids), poly(L-lactide-co-glycolides), poly(DL-lactide-co-glycolide), polyglycolides, polyanhydrides, polyorthoesters, polycaprolactones and their combinations and copolymers. These polymers also include those created from interlinked segments of D- and L-lactide, or combinations of these with DL-lactide.

Other preferred polymers include gelatin, albumin, fibrin, fibrinogen and collagen which are water-soluble and gellable in addition to being biodegradable.

Water-soluble polymers such as polyethylene glycol, polyvinyl alcohol, polyvinylpyrrolidone, polyoxyethylene-polypropylene block copolymers or other water-soluble polymers can be copolymerized with any of the polymers that can be used in this invention.

Where the application is such that there is no need for biodegradation of the polymer such as in oral, vaginal, rectal, topical, or transdermal administration, then a non-biodegradable polymer can be chosen. Such polymers can be chosen from the following classes of polymers without limitation such as ethyl celluloses, acrylates, methacrylates, pyrrolidones, polyoxyethylenes, polyoxyethylene-polypropylene copolymers, hydroxypropylmethyl celluloses, hydroxypropyl celluloses, methyl celluloses, polymethylmethacrylates, cellulose acetates and their derivatives, shellac, methacrylic acid based polymers more popularly known as EUDRAGITS, their copolymers and mixtures in different ratios. Mixtures of biodegradable and non-biodegradable polymers can also be used. Other classes of non-biodegradable polymers which are not described here but are known to those skilled in the art also fall within the scope of this invention.

In one of the embodiments of this invention, a mixture of polymers is used in the preparation of the gelled composition. The polymer mixture comprises one or more water-insoluble or water-soluble polymer(s) and at least one low melting polymer. If a water-insoluble polymer is used the low melting polymer must be capable of mixing with the insoluble polymer. The low melting polymer can be chosen from materials which melt at temperatures of less than 100° C., preferably less than about 80° C. The low melting polymer can be either water-soluble or insoluble. Preferably, the low melting water-soluble polymer is selected from polyethylene glycols (PEGs), polycaprolactones, polyoxyethylene-polyoxypropylene block copolymers, polyethylene oxides, and other materials which melt at temperatures of less than 100° C., preferably less than about 80° C. More preferably, the low melting water-soluble polymer is chosen from PEGs and polyethylene oxides. There is no limitation on the selection of the low melting polymer except that it should melt at a low temperature and be completely or partially miscible with the water-insoluble or water-soluble polymer.

The use of polymer blends allows the formation of polymers of different hydrophilic-hydrophobic characteristics with simple mixing without actually changing the polymer. Thus, polymers of two or more kinds can be simply blended and used in the preparation of the delivery system of the invention. The polymers can be mixed in any ratio from 100:0 to 0:100% w/w. The kinds of polymers to be blended, the actual percentages of the polymers and the ratios in which they are to be mixed will be readily apparent to a person skilled in the art of preparing polymeric drug delivery systems. For example, if a polymer mixture with greater hydrophilicity is required then a water-insoluble and water-soluble polymer are mixed and a higher percentage of the water-soluble polymer is used. If a more hydrophobic polymer mixture is required than a higher percentage of the water-insoluble polymer is used.

In another embodiment of the invention, only a single type of polymer is used. For example, a water-soluble low melting polymer such as polyethylene glycol or a water-insoluble low melting polymer such as polycaprolactone may be used alone.

There is no limitation on the kind of polymer which can be chosen as long as it is soluble in the solvent systems of this invention.

### The Biocompatible Organic Solvents

The solvents of this invention should be completely water-soluble and miscible with aqueous media in all proportions and include without limitation N,N′-dimethylacetamide (DMA), glycofural, dimethylsulfoxide (DMSO), N-methyl-2-pyrrolidone (NMP), water, 2-pyrrolidone, ethanol, propylene glycol, polyethylene glycol, glycerol, sorbitol, dimethylformamide (DMF), dialkylamides, caprolactam, glycerolformal, decylmethyl sulfoxide and other polar solvents, because of their exceptional solvating capability for the polymers described above, their non-volatility and their complete miscibility with water and with each other.

The viscosity of the polymer solution is governed by the type of polymer, concentration of the polymer and molecular weight of the polymer. A particular solvent or solvent composition should be chosen for each polymer to provide a polymer solution of optimum solubility and of optimum viscosity. When a drug will be incorporated into the polymer solution, the solvent used in the invention must provide a polymer solution with a high enough viscosity to carry a fairly high drug load but should not be too viscous for processing for the purposes of the invention. This is also true when a bioinactive agent is used. The choice of solvents and solvent systems for different polymers is within the scope of understanding for a person skilled in the art of making polymer based drug delivery systems.

In one of the embodiments of the invention, a mixture of water-soluble solvents is used to dissolve the polymer to provide a final composition of exceptional stability. Accordingly, mixtures of the water-soluble solvents in different ratios are used. It is also possible to use a mixture of solvents for the preparation of the polymer solutions for the purposes of dissolution of the biologically active substance or enhancing the rate of precipitation of the polymer upon contact with aqueous fluids.

### Polymer Concentrations

It is preferred to use polymer concentrations of between 1-90% w/w with respect to the solvent in the polymer phase. Even more preferably the polymer concentrations are in the range of 5-70% w/w. An even more optimum concentration is that between 10-60% w/w with respect to the solvent. The molecular weight of the polymer, copolymer or mixtures of polymers and their crystallinity will determine the solution viscosity. Thus a high molecular weight polymer will provide a solution of higher viscosity at a lower concentration when compared with a lower molecular weight polymer from the same class. Polymer solutions of concentrations of upto 60% w/w can be processed by raising the temperature of the polymer solution upto 25-75° C. Such concentrated polymer solutions of 10-60% w/w allow the delivery of higher loads of biologically active substances in smaller volumes of the final delivery system in contrast to the prior art compositions. Polymers concentrations up to 70% w/w can be processed by raising the temperature up to 95° C. Polymer concentrations of greater than 60% w/w to 90% w/w can be prepared by heating to 75-95° C. If a low melting polymer is used then the polymer solutions of greater than 60% w/w to 90% w/w can be processed at temperatures below 75° C.

The polymer solution will generally comprise 0.01-60%w/w of the total composition. More preferably the polymer solution will comprise 5-50%w/w and even more preferably 10-40%w/w of the total composition.

The use of the low melting polymer in this invention allows a reduction in the total amount of the solvent used for formulating the gelled dispersion. It is preferred to use a low melting polymer with a non low melting polymer in order to prepare polymer concentrations of 60% w/w to upto 90%. It is also possible to prepare a gelled polymeric dispersion using for example, a low melting polymer such as PEG 4000 or a liquid polymer such as PEG 200 to PEG 900 as the only solvent. Thus, a mixture of a low molecular weight poly(dl-lactide-co-glycolide) along with a PEG 4000 when heated and mixed in a ratio of 1:1 can form a gelled polymeric dispersion upon emulsification of the polymer melt into the continuous oil phase. A small amount of a solvent such as a liquid PEG could be added to reduce the viscosity of the polymer solution.

### The Biocompatible Oils

The oils used in this invention are biocompatible, nontoxic, nonirritant, and a non-solvent for the polymer. The oil is chosen from classes of oils which are allowed for pharmaceutical parenteral use. Such oils include without limitation various grades of animal oils such as whale oil or shark liver oil, or vegetable oils such as sesame seed oil, cottonseed oil, poppy seed oil, castor oil, coconut oil, canola oil, sunflower seed oil, peanut oil, corn oil, soyabean oil, or their fractionated counterparts such as capric-caprylic triglycerides and their salts with other acids. Preferably, the oil is chosen from super refined fixed vegetable oils such as sesame seed oil, soyabean oil, castor oil, fractionated coconut oil, poppy seed oil and such other pharmaceutically acceptable vegetable oils and their derivatives. Isopropyl myristate can also be used. Other classes of oils and their derivatives or mixtures of different oils in different proportions are known to those skilled in the art and also fall within the scope of this invention. There is no limitation to the kind of biocompatible oil chosen as long as it is gelled by the emulsifiers of the invention.

The biocompatible oil can comprise between 20-90% w/w of the total composition. More preferably the continuous oil phase will comprise 35-80% w/w of the total composition. Even more preferably the oil phase will comprise between 40-70% w/w of the total composition. Preferably, the concentration of polymer solution (discontinuous phase) with respect to the oil phase is 0.01 to 40% w/w.

### The Biocompatible Emulsifiers

The continuous oil phase contains from 5-70% w/w of the non-ionic emulsifiers sorbitan monostearate, sorbitan monopalmitate or a mixture thereof. The percentage of these non-ionic emulsifiers added to the oil phase will depend upon the amount of the emulsifier required to gel the continuous oil phase in the presence of the polymer solution. The higher the amount of the polymer solution that is to be emulsified the greater the amount of emulsifier is required. Also, a higher percentage of the emulsifier would impart additional stability to the gelled polymeric dispersion through an increase in the droplet stabilization. The determination of the percentage of the emulsifier required to form the gelled polymeric dispersion can be determined by a person skilled in the art of forming disperse systems.

The polymer solution can also optionally contain certain percentages of sorbitan monostearate, sorbitan monopalmitate or a mixture thereof to aid the stabilization of the dispersion to be formed.

Other emulsifiers that can be used in the polymer solution may be chosen from but are not limited to polysorbates, lecithins, other sorbitan esters of fatty acids, or other emulsifiers used in the formulation of disperse systems. These emulsifiers are used in concentrations of 0.1-60% w/w with respect to the polymer solution. More preferably the weight percentage of the emulsifier with respect to the polymer solution is between 5 and 50%

In addition, 0.001-70% w/w of other oil-soluble emulsifiers could be added to the oil phase to stabilize the polymer droplet-in-oil dispersion. Such emulsifiers include but are not limited to lecithins, sorbitan esters of fatty acids, polyoxyethylene esters of fatty acids, and other emulsifiers used in the formulation of disperse systems or their combinations in different ratios. The emulsifiers should be present in sufficient concentrations to stabilize the polymer droplet-in-oil dispersion. Even more preferably the concentrations are in the range 0.01-50% w/w with respect to the continuous oil phase. Other classes of emulsifiers or emulsion stabilizers known to those skilled in the art of making disperse systems and their combinations are also included without limitation.

The presence of a suitable hydrophilic emulsifier such as polysorbates, lecithins, polyethoxylated fatty acids and such other hydrophilic emulsifiers, in concentrations ranging from 0.01-10% w/w with respect to the oil phase along with the emulsifier which stabilizes the polymer droplet-in-oil effects the rate at which the continuous oil phase is emulsified and dissipates away from the injection site to allow the formation in-situ of the polymeric microcarriers from the novel dispersion. Thus, where a slow emulsifying dispersion is required, no or very little of the hydrophilic emulsifier is used. Other classes of hydrophilic emulsifiers or emulsion stabilizers known to those skilled in the art of making disperse systems and their combinations are also included without limitation.

### The Process of Manufacture of the Composition

The process of preparation of the dispersion of the invention comprises the steps of:

(a) dissolving a biocompatible polymer in a biocompatible water-soluble organic solvent or a mixture of solvents at an elevated temperature to form a polymer solution,

(b) separately dissolving a biocompatible emulsifier in a biocompatible oil at an elevated temperature to form a continuous oil phase,

(c) emulsifying the polymer solution as described in (a) above into the continuous oil phase as described in (b) above to form a polymer droplet-in-oil dispersion, and

(d) mixing the polymer droplet-in-oil dispersion and subsequently cooling it while mixing continuously, to obtain the final gelled dispersion.

The polymer solution at an elevated temperature (65-100° C.), is dispersed in the continuous oil phase at the same temperature, preferably, in a flow-through cell or a static mixer and with the aid of shear provided by high-speed homogenization, at speeds of 2000-25,000 rpm, probe sonication, high pressure homogenization or atomization through a spray nozzle under pressure of a compressed gas, or atomization through a ultrasonic nozzle. The temperatures of the oil and polymer phases can be chosen within 65-100° C. depending upon the stability of the oils, emulsifiers, the polymer in the solvent and if present, the biologically active or bioinactive agent.

It is preferable to inject the polymer solution into the oil phase through a narrow bore needle preferably a 15-25 gauge needle at a rate of 1-100 ml/minute. Such an injection procedure can be carried out through the use of a syringe-and-needle assembly or via the use of controlled positive displacement pumps such as peristaltic pumps, syringe pumps and the like.

The dispersion can be cooled either through continuous mixing while cooling to temperatures of 0-30° C. or by placing the dispersion at a low temperature of −20° C.

It is also possible to manufacture the dispersions at an elevated temperature (65-100° C.) and subsequently cool to refrigeration temperatures (2-8° C.) with continuous homogenization to achieve a product with enhanced content uniformity. The homogenization speed during this cooling step could be the same as that in the dispersion step or could be changed to a higher or lower speed. It is preferable that the homogenization speed be higher during the dispersion step and lower during the cooling step. It is also possible to rehomogenize the polymeric dispersions once they have been brought to refrigeration temperatures. The exact homogenization speeds to be used and the time for which homogenization should be carried out can be readily determined by a person skilled in the art of manufacturing disperse systems.

It is of course understood that the manufacturing process as described above could be readily extended to other forms of shear apart from high speed homogenization such as high pressure homogenization, microfluidization, colloid mill, triple roller mill, and such other methods of providing shear known in the art of manufacture of disperse systems. It is also possible to use a combination of the above mentioned procedures of providing shear. For example, a gelled composition could be prepared using high-speed homogenization as described above. This gelled composition could be used as a feed material for further high-pressure homogenization or microfluidization to further reduce the droplet size as desired. The various parameters including homogenization pressure, number of cycles, processing temperature and such other parameters which govern the efficiency of high-pressure homogenization or microfluidization would then govern the final outcome. Whatever the method or combination of methods used, the final outcome will be a gelled polymeric dispersion capable of forming the microcarrier delivery system of the invention.

The droplet size of the dispersion will determine the rate of extraction of the solvent and also the final particle size and shape of the microparticles achievable. The extraction of the solvent occurs when the polymer solution droplet comes in contact with the aqueous medium. The smaller the droplet size, the greater the surface area and hence the faster the rate of solvent extraction. A droplet size of 1-400 μm, preferably 5-150 μm, with greater than 40-60% of the droplets having an average size less than 100 μm, is desirable. The size can be varied by a person skilled in the art of manufacture of dispersions by the variation in the sizes of the homogenizer probes used, the speed of homogenization, the temperatures of both the phases, the polymer concentration in the organic solvent, the ratio of the discontinuous (polymer phase) to continuous (oil phase) phases, and such other parameters apparent to the person skilled in the art of microencapsulation, disperse systems and drug delivery and are all included herein by reference.

The gelled composition may be stored under refrigeration (2-8° C.) until further use.

### The Biologically Active Agent

The term drug, bioactive or biologically active agent as defined within the scope of this invention includes without limitation physiologically or pharmacologically active substances that act locally or systemically in a body. The terms drug, bioactive agent and biologically active agent are used interchangeably in the specification and claims. A body includes but is not limited to a human body or an animal body. Representative drugs and biologically active agents that can be used with the novel dispersions include, without limitation, peptide drugs, protein drugs, desensitizing agents, antigens, vaccines, anti-infectives, antibiotics, antimicrobials, antineoplastics, antitumor, antiallergenics, steroidal anti-inflammatory agents, analgesics, decongestants, miotics, anticholinergics, sympathomimetics, sedatives, hypnotics, antipsychotics, psychic energizers, tranquilizers, androgenic steroids, estrogens, progestational agents, humoral agents, prostaglandins, analgesics, antispasmodics, antimalarials, antihistamines, cardioactive agents, non-steroidal anti-inflammatory agents, antiparkinsonian agents, antihypertensive agents, beta-adrenergic blocking agents, nutritional agents, antivirals, DNA fragments, nucleic acids, genetic material, oligonucleotides, radioisotopes, or combinations of these classes of compounds. To those skilled in the art, other drugs or biologically active agents that can be released in an aqueous environment can be utilized in the described delivery system. Also, various forms of the drugs or biologically active agents may be used. These include, without limitation, forms such as uncharged molecules, molecular complexes, salts, ethers, esters, amides, and other chemically modified forms of the biologically active agent which are biologically activated when injected into a body.

### The Biologically Inactive Agent

The term biologically inactive agent as defined within the scope of this invention includes without limitation compounds and compositions such as lactic acid, glycerol, perfumes and antioxidants and other compounds and compositions useful in the preparation of compositions for cosmetic applications. The terms biologically inactive agent and bioinactive agent are used interchangeably in the specification and claims.

### The Drug Delivery System

An envisioned use of the novel gelled polymeric dispersion is to provide a novel drug-delivery system. Accordingly, in one embodiment, a bioactive agent is added to the polymer solution prior to emulsification. The drug can also be added as a solution or suspension. The drug in the polymer solution can be from 0.01-50% w/w with respect to the polymer in the polymer solution. This concentration of the drug is in respect to the polymer only and not with respect to both the polymer and the solvent. In some cases, the drug will also be soluble in the solvent, and a homogenous solution of polymer and drug will be available. In other cases, the drug will not be soluble in the solvent, and a suspension, emulsion or dispersion of the drug in the polymer solution will result. This suspension or dispersion can also be subjected to emulsification. In either case, upon administration of the novel dispersion of the invention, the solvent will dissipate and the polymer will solidify and entrap or encase the drug within the solid matrix to form the polymeric drug delivery system. Once the oil from the oil phase has dissipated away from the administration site to be absorbed into the body, the release of drug from the final formed solid implants will follow the same general rules for release of a drug from a monolithic polymeric device.

In order to provide an initial release of biologically active agent where required, the drug can also be added directly into the continuous oil phase either as a solution (where the drug is oil soluble) or as a suspension (where the drug is oil-insoluble), Preferably for such purposes, the drug could be added in concentrations of upto 1-50% w/w with respect to the oil phase.

The biologically active agent can be added to the polymer solution and/or the continuous oil phase, either as a solution or a suspension depending upon the solubilities of the drug in the two phases. Either way, the formation of the microcarrier delivery system from the composition and controlled release of the biologically active agent will follow.

The amount of drug or biologically active agent incorporated into the in-situ forming microcarrier delivery system depends upon the desired release profile, the concentration of drug required for a biological effect, and the length of time that the drug has to be released for treatment. There is no critical upper limit on the amount of drug incorporated into the polymer solution or the continuous oil phase except for that of an acceptable solution or dispersion viscosity for injection through a syringe needle. The lower limit of drug incorporated into the delivery system is dependent simply upon the activity of the drug and the length of time needed for treatment.

The release of drug from the delivery system can be affected by the oil phase concentration, the hydrophilicity of the continuous oil phase, the size and shape of the microcarriers, the rate of precipitation of the polymer to form the microcarriers, the loading of drug, the permeability factors involving the drug and the particular polymer, and the degradation of the polymer. Depending upon the drug selected for delivery, the above parameters can be adjusted by one skilled in the art of drug delivery to give the desired rate and duration of release.

The continuous oil phase can itself behave as a controlled release component because of the presence of the oil and the sorbitan esters. The rate of formation of the controlled release microcarrier delivery system in-situ can be manipulated by the presence or absence of the hydrophilic emulsifiers of the invention. When the hydrophilic emulsifiers are absent from the continuous oil phase or are present in low concentrations such as less than 0.1% w/w with respect to the oil phase, the rate of emulsification of the continuous oil phase is slow thus allowing the oil phase to act as a controlled release medium itself. The release of the biologically active agent will then occur through the combined mechanism of diffusion of the active agent through the oil phase along with the biodegradation and absorption of the different components of the composition. Thus, in such cases the drug release may be completed long before the composition is absorbed completely.

In the case of the use of biodegradable polymers, the microcarriers formed from the polymer system will slowly biodegrade within the body and allow natural tissue to grow and replace the implant as it disappears. Where water-soluble biodegradable polymers such as gelatin, polyethylene glycols, collagens, albumin and others are used the polymers will be absorbed into the body. For drug-delivery systems, the microcarriers formed from the polymer system will release the drug contained within its matrix at a controlled rate until the drug is depleted. With certain drugs, the polymer will degrade after the drug has been completely released. With other drugs such as peptides or proteins, the drug will be completely released only after the polymer has degraded to a point at which the non-diffusing drug now becomes exposed to the body fluids. In any case, the rate of release of the drug will be controlled by the rate at which the drug can diffuse out and/or the degradation rate of the polymer.

The rate of release of the biologically active agent from drug delivery systems formed from biodegradable polymers is governed by the water-solubility of the polymer, molecular weight of the polymer, the kind of polymer or copolymerization with other monomers, the crystallinity of the polymer used. The use of highly crystalline polymers such as those prepared from L-lactide or glycolide would give rise to a slower degrading polymer and hence a slower release profile. Copolymers of lactide and glycolide or the use of DL-lactide as against L-lactide give rise to polymers which are more hydrophillic and hence release the drug substance faster through a faster rate of biodegradation.

In the case of the use of non-biodegradable polymers, once the microcarriers are formed upon coming in contact with aqueous media the drug release will be determined based on the kind of polymer used. It is possible to achieve pH dependent release, diffusion controlled release or erosion controlled release through the selection of polymers with appropriate characteristics.

Bioinactive agents can be used with or in lieu of the drug, bioactive agent or biologically active agent in the drug delivery system in the same way as described above.

### The Mode of Administration

The dispersion of this invention may be part of a kit or device. A kit for the in-situ formation of microcarriers comprises:

(a) a pharmaceutical composition for providing an in-situ forming controlled release microcarrier delivery system, said composition being a gelled, syringeable droplet-in-oil dispersion comprising a biocompatible, biodegradable or non-biodegradable polymer in a water-soluble organic solvent and a biocompatible emulsifier in solution in a biocompatible oil, wherein the biocompatible emulsifier comprises sorbitan monostearate, sorbitan monopalmitate or mixture thereof wherein the concentration of said polymer in solution in said solvent, and of the emulsifier in solution in said oil are effective such that said dispersion when it comes into contact with an aqueous fluid forms said in-situ controlled release microcarrier delivery system; and,

(b) a device containing said pharmaceutical composition, said device having an inlet for the gelled dispersion, an ejector for expelling the gelled dispersion through an outlet into a site of a body such that the gelled dispersion can form a multitude of microcarriers in-situ at said site.

The compositions and kits of this invention can be used in the prevention or treatment of health disorders, diseases or medical conditions.

The preparation of this invention can be administered to the body by a syringe and needle assembly parenterally or by the use of a hard or soft gelatin capsule for oral, rectal or vaginal administration or as a creamy gel for topical administration. The formulation may also be administered via other pharmaceutically acceptable routes of administration.

Where the formulation is to be administered parenterally, the formulation will be filled into single-chambered prefilled syringes having preferably conventional 10-26 gauge needles, under continuous mixing.

It is also preferable to administer drug substances orally as multiparticulate formulations as compared to monolithic formulations because of known problems such as dose-dumping and its associated toxicities. To date, multiparticulate delivery systems are prepared by the use of techniques such as fluid-bed coating of drug-loaded non-pareil beads or as microencapsulated drugs filled into hard-gelatin capsules both of which are time consuming and expensive. The novel dispersions of this invention allow the formation of a polymeric microcarrier delivery system in-situ. Where such a use is intended, the novel dispersions can be filled into hard or soft gelatin capsules under mixing. Other additives required for oral drug delivery could be added. Where the patients have difficulty in swallowing the hard or soft gelatin capsules, the gelled composition could be formulated into a smooth suspension immediately before administration. This can be most readily accomplished by the addition of the gelled dispersion to a container containing a aqueous mixture containing a variety of excipients such as suspending agents, preservatives, coloring agents, flavoring agents and others, and subsequently shaking the mixture to form a smooth suspension.

Where the intended use of the gelled polymeric dispersion is for topical application, the dispersion can be formulated into a cream, paste or ointment and the like and can be filled into for example plastic or aluminum tubes or into wide-mouth jars from which the dispersion could be either squeezed out or applied with the use of an applicator.

The novel polymeric dispersions of the invention can also find use in vaginal delivery, intrauterine delivery, transdermal delivery and other routes of administration known to a person skilled in the art of administration of medications. Where the compositions are to be administered rectally or vaginally, it is preferable to formulate the compositions into suppositories or pessaries. This can be readily achieved by the addition of the gelled composition into a molten suppository base with subsequent cooling to room temperature after being poured into chilled molds. Alternatively, the gelled composition itself could be poured into chilled suppository molds under continuous mixing and cooled to room temperature. Upon administration, the formation of the microcarrier delivery system from the gelled composition occurs followed by the release of the biologically active agent or bioinactive agent incorporated into the composition.

The novel polymeric dispersions could also be used in other fields such as agriculture, controlled release of pesticides, in aquaculture, veterinary drug delivery and other fields. Whatever may be the route of administration and whatever may be the field of application the general principles of formation of the microcarrier delivery system from the novel gelled polymeric dispersions of the invention, will hold.

The following examples will further exemplify the invention in greater detail.

### EXAMPLES

The examples provided herein are only meant to exemplify the different aspects of the invention and are by no means meant to be limiting on the breadth and scope of the invention.

### Preparation 1

### General Method for Preparation of Polymers and Copolymers of Different Molecular Weights

Polymers and copolymers of lactic and glycolic acid were synthesized by the high temperature ring-opening polymerization of the lactide and glycolide cyclic dimers in the presence of stannous octoate as catalyst (Handbook of biodegradable polymers, Abraham J. Domb, Joseph Kost and David M. Weisman, Eds., Harwood Academic Publishers, 1997, Chapter 1, pages 3-28).

### Preparation 2

### Method of Synthesis of Copolymers of Water-Soluble and Insoluble Polymers

The copolymers of poly-DL-lactide and polyethylene glycol or poly-DL-lactide and polyvinyl pyrrolidone were prepared as per the general procedures described in U.S. Pat. No. 4,942,035 to Churchill et. al. In brief, DL-lactide, 15 g, was mixed with polyethylene glycol (PEG-4000), 5 g, or polyvinyl pyrrolidone, 4 g, and stannous octoate, 10 mg in toluene, in a 30 ml capacity test tube. The tube was purged with nitrogen, sealed and kept in an oil bath at 160° C. for 5 hours. Subsequently, the tubes were opened and the molten copolymers were poured in a tray lined with aluminum foil. The polymer was allowed to solidify, suitably milled and stored in a sealed container at −20° C. till use.

### Techniques used for the Characterization of the Novel Polymer Systems

A. Syringeability and Microcarrier Formation

Syringeability and microcarrier formation from the novel systems was determined by filling the formulations into glass syringes fitted with needles of various gauges, ranging from 14-23 gauge, and injecting the formulation into glass vials containing pH 7.0 phosphate buffer containing 0.02% Tween 80 and 0.02% sodium azide at 37° C., hereinafter stated to be the “aqueous medium”. The tubes were then capped and placed in an orbital shaker at 37° C. and mixed at 100 oscillations per minute.

Syringeability is described as the smallest bore needle through which the formulations can be delivered with ease. Microcarrier formation is defined as the formation of a uniform dispersion within a maximum time period of 24 hours with the absence of any lumps or aggregates when observed visually.

B. Particle Size Measurement

The gelled compositions were filled into glass syringes fitted with 18 gauge needles and approximately 0.5-1.0 g of the gelled compositions were injected into glass tubes containing 10 ml of pH 7.0 phosphate buffer containing 0.02% Tween 80 and 0.02% sodium azide. The tubes were capped and placed in an orbital shaker at 37° C. and mixed at 100 oscillations per minute for 24 hours. The sizes of the formed microcarrier dispersions were measured using a Malvern particle size analyzer by laser light scattering.

C. Drug Release from the Novel Systems

The novel gelled polymeric dispersions (0.5 g) were injected using syringes attached with 18 gauge needles followed by the addition of 5 ml of the release medium into pieces of dialysis tubing tied at one end (SIGMA, molecular weight cut-off=12,000 D). The other end of the sacs were tied with threads and the sacs were placed into screw-capped glass tubes containing 15 ml of pH 7.0 phosphate buffer containing 0.02% w/v Tween 80 and 0.02% w/v sodium azide. The tubes were placed in a reciprocating incubator-shaker maintained at 37° C. with an oscillation speed of 100 oscillations per minute. At different sampling points post-initiation of the study, the release medium was removed from the tube and replaced with fresh medium. The amount of drug released into the medium was assayed by HPLC.

The actual amount of the polymer-drug solution incorporated in the final formulation was taken as the basis for the calculation of drug release and encapsulation efficiencies. The amount of biologically active agent entrapped within the particles was determined by the difference in the actual amount of drug incorporated in the final formulations during processing and the amount released in one day.

### Novel Gelled Polymeric Dispersions and the Polymer Systems Formed from These Dispersions

### Preparation of a Gelled Polymer-in-Oil Dispersion Containing a Highly Water-Soluble Peptidic Biologically Active Agent

### Example 1

### Part A

Poly(DL-lactide-co-glycolide) with a Mw of 13,000 D, 1 g, was dissolved in DMSO (Fluka, 2.3 g) aided by gentle heating to 65-70° C. to form a polymer solution of a 30%w/w concentration. To this solution 120 mg of leuprolide acetate was added to form a 10% w/w solution of the drug with respect to the polymer. The polymer solution was injected using a syringe attached with a 18 gauge needle into 10 g of a continuous oil phase comprising a 20% w/w solution of sorbitan monostearate (Arlacel 60, ICI Ltd.) in super refined sesame seed oil (Croda) maintained at a temperature of 70-75° C., accompanied by high speed homogenization at 13,000 rpm, for 3 minutes. The resulting polymer droplet-in-oil dispersion was cooled to room temperature with continuous mixing to obtain an opaque mass with a gel like consistency, which did not flow. The gel was stored under refrigerated conditions until further use.

The gel was smooth to the touch with an absence of any gritty particles. Observation of the gel under a microscope revealed discrete distorted blue colored droplets of the discontinuous phase dispersed within the continuous oil phase.

### Formation of the Polymer System of the Invention from the Novel Gelled Polymer-in-Oil Dispersion

### Example 1

### Part B

The novel gelled polymeric dispersion obtained in Example 1, Part A was filled into a glass syringe attached with a 18 gauge needle and was easily injected into a beaker containing the aqueous medium being mixed gently with the aid of a magnetic stirrer. The gel structure broke down and fine, discrete particles of the polymer entrapping the leuprolide acetate of an average particle size of 46.85 μm, settled to the bottom of the beaker.

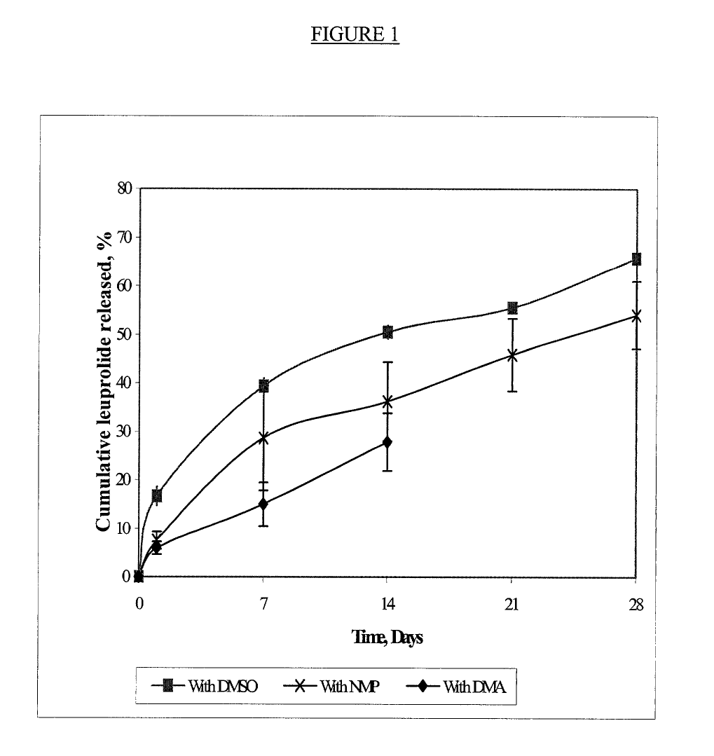

A drug release study indicated leuprolide acetate release in a controlled fashion with a burst effect of 16.67% at 24 hours (FIG. 1). The remaining drug being entrapped within the formed particles and released 60% over 28 days.

### Comparative Example 1

Solutions of a poly(DL-lactide-co-glycolide) with a Mw of 13,000 D in NMP, DMSO or DMA were prepared at concentrations of 30% w/w as per the procedure described in U.S. Pat. No. 6,143,314 to Chandrashekhar et. al. To these solutions leuprolide acetate was added in a concentration of 10% w/w with respect to the polymer. The presence of liquid droplets could not be confirmed when observed under a microscope.

The polymer drug solutions were dropped into the aqueous medium. In each case, a single large globule was observed which slowly formed a rigid monolithic implant. The formation of discrete particles could not be confirmed. This indicates that a solution of a polymer in a water-soluble organic solvent alone is not capable of forming discrete microcarriers upon coming in contact with an aqueous medium.

### Example 2

### Preparation of a Gelled Polymer-in-Oil Dispersion Containing a Water-Insoluble, Oil-Insoluble and Solvent-Insoluble Biologically Active Agent

A gelled dispersion was prepared by emulsifying a 40%w/w solution of poly-DL-lactide-co-glycolide copolymer (Comonomer ratio 75:25, inherent viscosity=0.15 dl/g, Birmingham Polymers Inc., USA) in DMSO, containing red iron oxide (10.78 mg), into the oil phase, comprising 10g of a 20% w/w solution of Arlacel 60 in sesame seed oil as per the procedure of Example 1. The dispersion was syringeable, forming discrete red colored particles upon being put into contact with an aqueous medium.

### Examples 3-5

### Effect of Using Different Water-Soluble Organic Solvents on the Formation and Characteristics of the Novel Gelled Polymeric Dispersions Containing a Water-Soluble Peptidic Biologically Active Agent such as Leuprolide Acetate

Gelled polymeric dispersions were prepared with a poly(DL-lactide-co-glycolide) polymer (comonomer ratio 75:25 mole %, Mw=13,000 D) in DMA, DMSO, and NMP, respectively, at polymer concentrations of 40% w/w in the solvents and containing leuprolide acetate, 10% w/w with respect to the polymer. The further gel formation and analyses were as per Example 1.

The gelled dispersions prepared with DMA, DMSO and NMP were all easily syringeable through a 18 gauge needle and formed discrete microcarriers of average sizes of 19.44 μm, 46.85 μm and 23.09 μm, respectively within 30 minutes upon coming in contact with an aqueous medium. The gelled dispersions were physically stable for 21 days at 2-8° C. without any signs of phase separation on visual observation.

A drug release study indicated burst effects of 5.98%, 16.67% and 7.55% respectively of leuprolide acetate from the novel gelled dispersions prepared from DMA, DMSO and NMP within 24 hours with the remaining drug being released in a controlled fashion over more than one month (FIG. 1).

### Examples 6-8

### Effect of Using Different Water-Soluble Organic Solvents on the Formation and Characteristics of the Novel Gelled Polymeric Dispersions Containing a Water-Insoluble and Oil-Insoluble but Solvent-Soluble Biologically Active Agent such as Paclitaxel

A gelled dispersion was prepared by emulsifying a 40% w/w solution of poly DL-lactide-co-glycolide copolymer (comonomer ratio 75:25 mole %, Birmingham Polymers Inc. USA), in DMSO, DMA or NMP and containing paclitaxel, 10% w/w with respect to polymer into a continuous oil phase comprising 5.0 g Arlacel 60, 0.4 g Tween-80 in 14.6 g sesame seed oil, and processed as described in Example 1.

All three gelled dispersions were easily syringeable through a 18 gauge needle and formed discrete microcarriers of an average size of 40 μm, 48 μm and 63 μm, respectively, within 30 minutes upon coming in contact with an aqueous medium. The gelled dispersions were physically stable for 21 days at 2-8° C. without any signs of phase separation on visual observation.

### Examples 9-10

### Formation and Characteristics of the Novel Gelled Polymeric Dispersions Using Mixtures of a Water-Insoluble Biodegradable Polymer such as a Poly DL-Lactide-Co-Glycolide Copolymer and a Water-Soluble Polymer such as Polyethylene Glycol

The novel gelled polymeric dispersions were prepared by using mixtures of a poly DL-lactide-co-glycolide copolymer (comonomer ratio 75:25 mole %, Birmingham Polymers Inc. USA) and a polyethylene glycol (Mw=4000), in ratios of 4 to 3 and 4 to 4.5. The leuprolide acetate loading was 10% w/w with respect to the copolymer, and the procedure of Example 1 was followed for preparation of the gels.

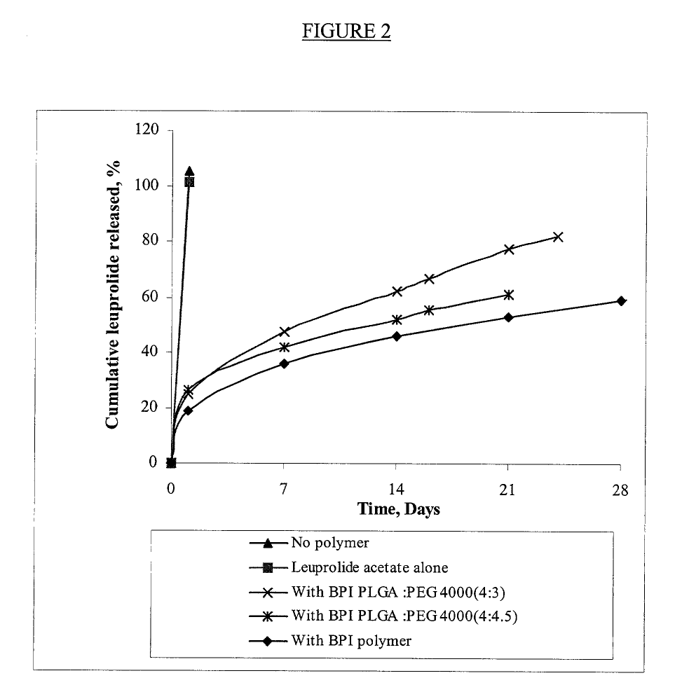

The gels were easily syringeable through an 18 gauge needle and formed discrete particles within 30 minutes upon coming in contact with an aqueous medium. The burst effect was 25% over 24 hours with the rest of the drug being entrapped within the formed particles (FIG. 2). The gels were physically stable at 2-8° C. for over 2 months.

This example demonstrates that the release of biologically active agents can be modified through the use of simple mixtures of a water-insoluble biodegradable polymer such as a poly DL-lactide-co-glycolide copolymer and a water-soluble polymer such as polyethylene glycol.

### Example 11

### Impact of the use of Mixtures of Water-Soluble Organic Solvents such as DMA and PEG 400 on the Formation and Physical Stability of the Novel Gelled Polymeric Dispersions—Gelled Dispersions Containing Paclitaxel

A gelled dispersion containing paclitaxel was prepared as follows. A poly-DL-lactide-co-glycolide polymer (Comonomer ratio 75:25, inherent viscosity=0.15 dl/g, BPI, USA) was dissolved in a solvent phase comprising of DMA: PEG 400 (25:75% w/w) by heating at 70° C. on an oil bath to make a 40% w/w polymer solution. Paclitaxel, 10% w/w, respectively with respect to the polymer was added to the polymer solution held at 70-85° C. and mixed till dissolved. This solution was then emulsified into an oil phase comprising of Arlacel-60, 2.5 g and Tween 80, 0.2 g, in 7.5 g sesame seed oil and held at 70° C., aided by homogenization at 11,000 rpm speed using a Ika Ultra-Turrax T-25-basic homogenizer. The homogenization was continued even during the cooling phase till the gel formation took place.

This gelled dispersion was easily syringeable through an 18 gauge needle and readily (within 5-7 minutes) formed particles upon coming in contact with the aqueous medium. The gelled dispersion contained 97.4±0.88% paclitaxel of the label claim. (Label claim indicates how much drug was added into the product during manufacture. (10% w/w with respect to the polymer) and an extremely low percent RSD (Relative standard deviation of 10 analyses) value of 0.901%. The dispersion was stable at 25-30° C. for at least 8 hours and for more than 2 months at 2-8° C.

### Examples 12-13

Similarly, gelled dispersions containing 25 and 50% w/w paclitaxel with respect to the polymer were also prepared. These gels were easily syringeable through 18 gauge needles and readily (within 5-7 minutes) formed particles upon coming in contact with the aqueous medium. The gelled dispersions contained 98.09±0.86% and 97.06±1.08%, paclitaxel respectively with respect to the label claims (25 and 50% w/w with respect to the polymer) and extremely low percent RSD values of 0.878% and 1.113%, respectively. The dispersions were stable at 25-30° C. for at least 8 hours and for more than 2 months at 2-8° C.

### Example 14

### In vivo Controlled Release of Paclitaxel from the Gelled Polymeric Dispersion when Administered Subcutaneously

The novel gelled polymeric dispersions containing 10% and 50% w/w paclitaxel respectively with respect to the polymer, prepared in Examples 11 and 13 were injected subcutaneously into female Wistar rats at a dose of 30 mg/kg body weight, using a 2 cc plastic syringe attached with a 18 gauge needle. Blood samples were withdrawn periodically, retroorbitally, and the plasma from the blood samples was recovered by routine procedures.

Briefly, the blood samples were collected in Eppendorf polypropylene tubes containing heparin (2-3 drops/ml of blood, 25,000 IU heparin/5 ml) and the tubes were centrifuged at 3000 rpm at 10° C. for 30 minutes. The plasma was separated into clean and sterile tubes and stored at −40° C. till further processing.

The paclitaxel content in the plasma samples was analyzed by a sensitive LC/MS/MS method. Briefly, paclitaxel from the plasma samples was extracted by solid phase extraction using Oasis HLB cartridges equilibrated with methanol on a Waters vacuum manifold. The paclitaxel was eluted using methanol, the solution was evaporated to dryness and reconstituted in a mixture of distilled water and acetonitrile. Paclitaxel was quantified on a LC/MS/MS (Micromass Quattro II) equipped with a HP 1100 HPLC. A C8 column (100 mm×2.1 mm, 5 μm) at ambient temperature was used with a run time of 4 minutes. The mobile phase employed was ammonium acetate buffer and acetonitrile in the ratio 25:75% v/v.

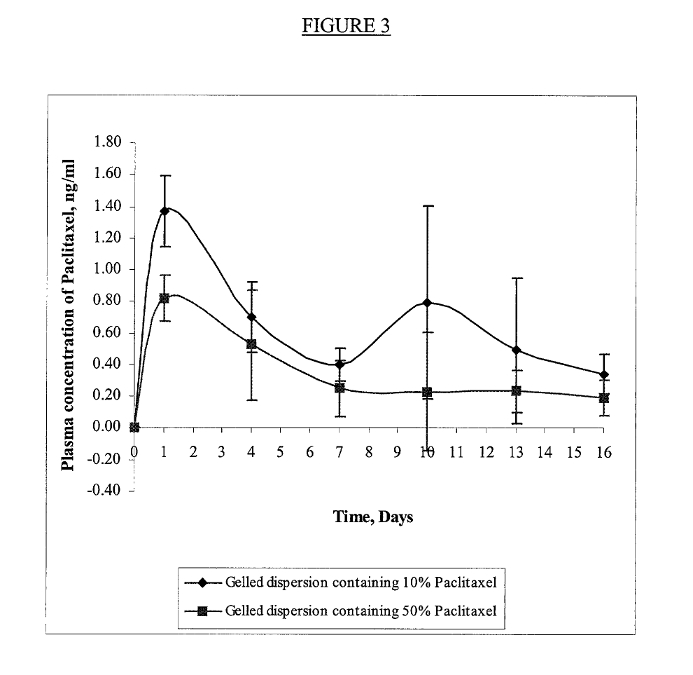

FIG. 3 shows a plot of the plasma profiles of paclitaxel when administered in the gel formulations. The drug was eliminated from the body with plasma elimination half-lives of 6.25 and 5 days respectively, when compared with the value of 1.1 hours when administered intravenously as a solution, reported in the literature (Alex Sparreboom, Olaf van Tellingen, Willem J. Nooijen and Jos H. Beijnen; (1998), “Preclinical pharmacokinetics of paclitaxel and docetaxel”, Anti-cancer Drugs, 9: 1-17).

This example demonstrates that the novel gelled polymeric dispersion is capable of providing controlled release of a biologically active agent such as paclitaxel over a prolonged period of time.

### Example 15

### Impact of the use of Mixtures of Water-Soluble Organic Solvents such as DMA and PEG 400 on the Formation and Physical Stability of the Novel Gelled Polymeric Dispersions—Gelled Dispersions Containing Leuprolide Acetate

A gelled dispersion was prepared as described in Example No. 11, using poly DL-lactide-co-glycolide copolymer (Comonomer ratio 75:25, Purac Polymers Inc., USA), 40% w/w solution in a solvent phase comprising of 25:75% w/w DMA: PEG 400, and containing leuprolide acetate, 10% w/w with respect to polymer.

The gelled dispersion was syringeable through a 20 gauge needle, formed discrete particles within 10 minutes upon coming in contact with the aqueous medium and was stable at room temperature for 21 days.

### Example 16

### In vivo Efficacy of a Novel Gelled Polymeric Dispersion Containing Leuprolide Acetate

The novel gelled polymeric dispersion containing leuprolide acetate prepared in Example 11 was filled into 2 cc plastic syringes attached with 18 gauge needles. The dispersions were injected intramuscularly into the thigh muscles of male Sprague-Dawley rats at a dose of 3 mg leuprolide acetate per kg body weight per animal. A placebo gel was administered as a control. Blood samples were obtained retroorbitally and the serum was collected according to routine procedures.

Briefly, the blood samples were collected into Eppendorf polypropylene tubes and held at 22-25° C. for 1 hour. The tubes were subsequently centrifuged at 3000 rpm at 10° C. for 30 minutes and the separated serum was collected into clean tubes and stored at −40° C. until further analysis. Serum testosterone levels were monitored by using an immunofluorescence method against testosterone standards.

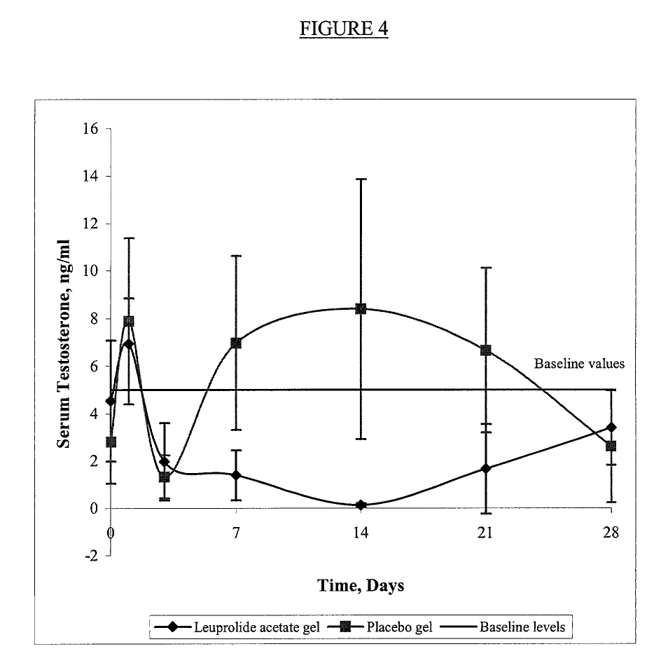

The data demonstrate the rapid and prolonged suppression of serum testosterone levels below the baseline levels for at least 28 days in the animal model when compared with the animals administered the placebo control (FIG. 4). The formulation thus provides controlled release of a highly water-soluble peptide over extended periods of time, in-vivo.

### Example 17

### Impact of the use of Mixtures of Water-Soluble Organic Solvents such as DMA and PEG 400 and Mixtures of a Water-Insoluble Biodegradable Polymer such as a Poly DL-Lactide-Co-Glycolide Copolymer and a Water-Soluble Polymer such as Polyethylene Glycol on the Formation and Physical Stability of the Novel Gelled Polymeric Dispersions—Gelled Dispersions Containing Paclitaxel

A gelled polymeric dispersion was prepared as described in Example 11 with a PLG copolymer (comonomer ratio 75:25 mole %, Birmingham Polymers Inc. USA, inherent viscosity 0.15 dl/g) dissolved in a solvent system comprising PEG 4000, PEG 400 and DMA (in 75: 6.4: 18.6% w/w proportion) by heating at 80-85° C. to make a 40% w/w solution. Paclitaxel, 10% w/w with respect to the PLG polymer was added to the polymer solution.

The gelled dispersion was syringeable through a 18 gauge needle, formed particles in 5-7 minutes on contact with an aqueous medium and was stable at 25-30° C. for 43 days.

### Example 18

### Effect of Varying the Concentration of the Emulsifier on the Physical Stability of the Gelled Polymeric Dispersion

The novel gelled polymeric dispersions containing paclitaxel, 10% w/w with respect to the polymer were prepared as per Example No. 11, but, with the use of Arlacel-60 at concentrations of 20, 25, 30, 35% w/w with respect to the oil phase.

The gelled dispersions were syringeable through 18 gauge needles, formed discrete particles within 5-7 minutes upon coming in contact with the aqueous medium and was stable at room temperature for 43 days.

It is thus possible to use combinations of the water-soluble solvents, polymer mixtures and different concentrations of the emulsifiers of the invention to produce gelled polymeric dispersions with exceptional stability at temperatures of 25-30° C. and capable of rapidly in-situ forming the microcarrier controlled delivery system.

The following examples further demonstrate the preparation of the novel gelled compositions with other polymers and drug substances.

The formation and characteristics of the novel gelled polymeric dispersions using different water-insoluble biodegradable polymers.

### Example 19

A gelled dispersion was prepared as per Example No. 11 with a poly-DL-lactide-co-glycolide polymer (PLG-36, comonomer ratio 75:25 mole %, MW=8566 D) dissolved in PEG-400 to form a 47% w/w solution. The gelled dispersion was syringeable through a 22 gauge needle, formed discrete particles within 10 minutes upon coming in contact with the aqueous medium and was stable at room temperature for 42 days.

### Example 20

A solution of poly-L-lactic acid (weight average molecular weight 6500), 2.0 g, and lidocaine hydrochloride, 0.2 g, in 4.6 g DMA, was injected into 20 g of oil phase comprising of Arlacel 60, 25% w/w, Tween 80, 2% w/w in sesame seed oil. The processing was carried out as per Example 1. The gelled dispersion was syringeable and formed discrete particles when injected into an aqueous medium at 37° C.

### Example 21

Example 20 was repeated but with chlorpheniramine maleate as the biologically active agent and NMP as the solvent. The processing was carried out as per Example 1. The gelled dispersion was syringeable and formed discrete particles when injected into an aqueous medium at 37° C.

### Example 22

A gelled dispersion was prepared using poly(DL-lactide-co-glycolide) copolymer (comonomer ratio 46:54, Mw 8546), 1 g, olanzapine, 0.1 g, dissolved in NMP, 2.3 g, to form the polymer phase. The polymer phase was emulsified into an oil phase comprising 2.5 g sorbitan monostearate (Arlacel 60), 0.2 g Tween 80 and 7.5 g sesame seed oil. The gelled dispersion was syringeable and formed discrete particles when injected into an aqueous medium at 37° C.

### Example 23

A gelled dispersion was prepared using a poly-DL-lactide polymer, 1 g, chlorpheniramine maleate, 0.1 g, dissolved in DMA, 2.3 g, to form the polymer phase. The polymer phase was emulsified into an oil phase comprising 2.5 g sorbitan monostearate (Arlacel 60), 0.25 g Tween 80 and 7.33g sesame seed oil. The gelled dispersion was syringeable and formed discrete particles when injected into an aqueous medium at 37° C.

### Example 24

A gelled dispersion was prepared using a 30% w/w solution of poly(DL-lactide-co-glycolide) (Mw=11,000), in NMP containing trimethoprim, 40 mg, and sulfamethoxazole, 200 mg. The polymer phase, 3.5 g was added to 10 g of the oil phase containing 25% w/w Arlacel 60 and 2% w/w Tween 80 and processed as per Example 1.

The gelled dispersion was spread on the forearm of a volunteer using a stainless steel spatula and had good spreadability.

### Example 25

A solution of poly L-lactic acid, (M. Wt. 785), 3 g, in DMSO, 1 g was injected into 20 g of oil phase comprising of 25% w/w Arlacel 40, in soya oil. Upon cooling, orange oil was added as a fragrance.

The polymer ‘cream’ so formed had easy spreadability on human skin and caused no irritation. The oil phase disappeared rapidly leaving behind a polymeric film on the skin. This has application in delivering lactic acid to the skin for its well documented ‘anti aging’ effect.

### Example 26

A solution of the poly-DL-lactide-co-PEG copolymer synthesized in Preparation 2, 2 g, and felodipine, 10% w/w with respect to the polymer, in DMA, 4.6 g, was injected into the oil phase comprising 5 g sorbitan monostearate (Arlacel 60), 0.4 g Tween 80 and 14.7 g sesame seed oil. and the processing was carried out as per Example 1. The gelled dispersion was syringeable and formed discrete particles when injected into an aqueous medium at 37° C.

### Example 27

A solution of poly-DL-lactide-co-vinylpyrrolidone copolymer synthesized in Preparation 2, 2 g, and felodipine 10% w/w with respect to the polymer, in DMSO, 4.6 g, was injected into the oil phase comprising 5 g sorbitan monostearate (Arlacel 60), 0.4 g Tween 80 and 14.7 g sesame seed oil. and the processing was carried out as per Example 1. The gelled dispersion was syringeable and formed discrete particles when injected into an aqueous medium at 37° C.

### Example 28

A solution of a poly-(DL-lactide-co-glycolide) (weight average molecular weight 11,031; comomomer ratio 72.04: 27.95), and octreotide acetate 10% w/w with respect to the polymer, in 2.3 g of DMSO was injected into the oil phase comprising 2.5 g sorbitan monostearate (Arlacel 60), 0.2 g Tween 80 and 7.3 g sesame seed oil and the processing was carried out as per Example 1. The gelled dispersion was syringeable and formed discrete particles when injected into an aqueous medium at 37° C.

### Example 29

A solution of a poly-(DL-lactide-co-glycolide) (weight average molecular weight 11,031; comomomer ratio 72.04: 27.95), and goserelin acetate 10% w/w with respect to the polymer, in 2.3g DMSO was injected into the oil phase, 10 g, comprising 25% w/w Arlacel 60 and 2% w/w Tween 80 in sesame seed oil. The processing was carried out as per Example 1. The gelled dispersion was syringeable and formed discrete particles when injected into an aqueous medium at 37° C.

### Example 30

A gelled dispersion was prepared as described in Example 11, using a PLG copolymer, 40% w/w solution in a solvent phase comprising 25:75% w/w DMSO: PEG 400, and containing olanzapine, 10% w/w with respect to polymer, to form the polymer phase. The gelled dispersion was syringeable through a 18 gauge needle, formed discrete particles within 10 minutes upon coming in contact with the aqueous medium and was stable at room temperature for at least 8 hours.

### Example 31

A gelled dispersion was prepared as described in Example 11, using a PLG copolymer, 40% w/w solution in a solvent phase comprising 25:75% w/w NMP: PEG 400, and containing felodipine, 10% w/w with respect to polymer, to form the polymer phase. The gelled dispersion was syringeable through a 18 gauge needle, formed discrete particles within 10 minutes upon coming in contact with the aqueous medium and was stable at room temperature for at least 8 hours.

### Example 32

A gelled dispersion was prepared as described in Example 11, using a PLG copolymer, 40% w/w solution in a solvent phase comprising of 25:75% w/w NMP: PEG 400 and containing Captopril, 10% w/w with respect to polymer, to form the polymer phase. The gelled dispersion was syringeable through a 18 gauge needle, formed discrete microcarriers within 10 minutes upon coming in contact with the aqueous medium and was stable at room temperature for at least 8 hours.

The formation and characteristics of the novel gelled polymeric dispersions using different water-soluble biodegradable polymers.

### Example 33

A solution of polyvinyl pyrrolidone, (Kollidon K25), 2.0 g, and olanzapine, 0.2 g, in 3 g DMA, was injected into 20 g of oil phase comprising of Arlacel 60, 25% w/w, Tween 80, 2% w/w in sesame seed oil. The processing was carried out as per Example 1. The gelled dispersion was syringeable and formed discrete particles when injected into an aqueous medium at 37° C.

### Example 34

Example 33 was repeated but using polyvinyl pyrrolidine (kollidon K-90 BASF), 1 g, olanzapine, 0.1 g, dissolved in DMSO, 4 g, to form the polymer phase. The novel gelled dispersion was further processed as per Example 1 with 10 g of the oil phase. The gelled dispersion was syringeable and formed discrete particles when injected into an aqueous medium at 37° C.

### Example 35

A gelled dispersion was prepared as described in Example 11, using polyvinyl pyrrolidone (Kollidon K25 BASF), 30% w/w solution in a solvent phase comprising of 25:75% w/w DMA: PEG 400, and containing indomethacin, 10% w/w with respect to polymer, to form the polymer phase. The gelled dispersion was syringeable through a 18 gauge needle, formed discrete particles within 10 minutes upon coming in contact with the aqueous medium and was stable at room temperature for 8 days.

### Example 36

A gelled dispersion was prepared as described in Example 11, using polyvinyl pyrrolidone (Kollidon K25 BASF), 30% w/w solution in a solvent phase comprising of 25:75% w/w DMSO: PEG 400, and containing olanzapine, 10% w/w with respect to polymer, to form the polymer phase. The lot was processed as per example 8 but the Arlacel-60 concentration was increased to 35% w/w in the oil phase. The gelled dispersion was syringeable through a 18 gauge needle, formed discrete particles within 10 minutes upon coming in contact with the aqueous medium and was stable at room temperature for at least 8 days.

### Example 37

A gelled dispersion was prepared as described in Example 11, using 79.6% w/w PEG 4000 in PEG 400 and paclitaxel 10% with respect to the polymer. The gelled dispersion was syringeable through a 18 gauge needle, formed discrete particles within 10 minutes upon coming in contact with the aqueous medium and was stable at room temperature for 45 days.

### Example 38

A gelled dispersion was prepared as described in Example 11, using 40% w/w gelatin in water and paclitaxel 10% with respect to the polymer. The gelled dispersion was syringeable through a 18 gauge needle, formed discrete particles within 10 minutes upon coming in contact with the aqueous medium and was stable at room temperature for 11 days.

### Example 39

A solution of povidone iodine, 2.2 g, in 3 g DMA was emulsified into 20 g of sesame seed oil containing Arlacel 60, 25% w/w and the gel was prepared as per the procedure of Example 1. The gelled dispersion was physically stable at 2-8° C. for 2 months.

The gelled dispersion was filled in a collapsible aluminum tube and squeezed into a release medium at room temperature. The gel readily dispersed into discrete particles. The gel was applied topically on human skin. The oily component disappeared rapidly leaving behind fine povidone iodine spots spread over the area of application indicating the formation of discrete polymer droplets.

The formation and characteristics of the novel gelled polymeric dispersions using different water-insoluble non-biodegradable polymers

### Example 40

A solution of Eudragit E-100, (a methylacrylic acid copolymer, Rohm Pharma) 2.0 g, and pseudoephedrine HCl, 0.2 g, in 4.6 g of DMA, was injected into 20 g of oil phase comprising Arlacel 60, 25% w/w, in sesame seed oil. The processing was carried out as per Example 1. The gelled dispersion was syringeable through a 18 gauge needle and formed discrete particles when injected into the aqueous medium at 37° C.

The gelled dispersion, 0.55 g, was filled into size zero, colorless, transparent, hard gelatin capsules and the capsules were sealed. It formed discrete particles when added into 0.1 N HCl maintained at 37° C.

### Example 41

A solution of Eudragit E-100, 2.0 g, and felodipine, 0.2 g, in 4.6 g DMA, was injected into 20 g of oil phase comprising of Arlacel 60, 25% w/w. The processing was carried out as per Example 1. The gelled dispersion was filled in a 10-ml glass syringe fitted with an 18-gauge needle. The gelled dispersion was syringeable and formed discrete particles when injected into the aqueous medium at 37° C.

The gelled dispersion was filled into size zero, colorless, transparent, hard gelatin capsules. It formed discrete particles when added into 0.1 N HCl at 37° C.

### Example 42

A suspension of felodipine, 0.5 g, in SURELEASE (a commercial aqueous polymeric dispersion of ethyl cellulose), 5 g, was injected into 20 g of oil phase comprising of Arlacel 60, 25% w/w, Tween 80, 2% w/w in sesame seed oil. The processing was carried out as per Example 1. The gelled dispersion was syringeable and formed discrete particles when injected into the aqueous medium at 37° C.

### Example 43

A solution of shellac, 2.0 g, and pseudoephedrine HCl, 0.2 g, in 3 g DMA, was injected into 20 g of oil phase comprising of Arlacel 60, 25% w/w, Tween 80, 2% w/w in sesame seed oil. The processing was carried out as per Example 1. The gelled dispersion was syringeable and formed discrete particles when injected into the aqueous medium at 37° C.

### Example 44

A gelled dispersion was prepared using a 1:1 parts w/w blend on Eudragit E-100 (a methlacrylic acid copolymer (Rohm Pharma)) and Eudragit L-100 (a methlacrylic acid copolymer Rohm Pharma) in DMSO using indomethacin as the model drug. The total polymer concentration was 20% w/w of the polymer phase and indomethacin was added in 2% w/w concentration with respect to the polymer phase. The polymer phase, 10 g was added to 20 g of the oil phase and processed as per Example 1. The oil phase comprising 5 g sorbitan monostearate (Arlacel 60), 0.4 g Tween 80 and 14.6 g sesame seed oil. The gelled dispersion was syringeable and formed discrete particles when injected into the aqueous medium at 37° C.

The formation and characteristics of the novel gelled polymeric dispersions using different water-soluble non-biodegradable polymers

### Example 45

A gelled dispersion was prepared as described in Example 11, using Lutrol F-68, (a polyoxyethylene-polyoxypropylene block copolymer BASF) 30% w/w solution in a solvent phase comprising of 25:75% w/w DMA: PEG 400 and containing chlorpheniramine maleate, 10% w/w with respect to polymer, to form the polymer phase. The gelled dispersion was syringeable through a 18 gauge needle, formed discrete particles within 10 minutes upon coming in contact with the aqueous medium and was stable at room temperature for at least 8 hours.

### Example 46

A solution of hydroxypropylmethylcellulose, 2.0 g, and felodipine 0.2 g, in 3 g DMA, was injected into 20 g of oil phase comprising of Arlacel 60, 25% w/w. The processing was carried out as per Example 1. The gelled dispersion was stable at 2-8° C. for 2 months, was syringeable through a 18 gauge needle and formed discrete particles when injected into the aqueous medium at 37° C.

### Example 47

A solution of betacyclodextrin, 2.0 g, and felodipine, 0.2 g, in 4.6 g DMA, was injected into 20 g of oil phase comprising of Arlacel 60, 25% w/w, Tween 80, 2% w/w in sesame seed oil. The processing was carried out as per Example 1. The gelled dispersion was syringeable and formed discrete particles when injected into the aqueous medium at 37° C.

### Example 48

Lutrol F-68 (a polyoxyethylene-polyoxypropylene block copolymer BASF), 1 g, and terbutaline sulphate, 0.2 g, were dissolved in DMSO, 2.3 g, to form the polymer phase. The novel gelled dispersion was further processed as per Example 1 with the oil phase comprising 2.5 g sorbitan monostearate (Arlacel 60), 0.22 g Tween 80 and 7.3 g sesame seed oil. The gelled dispersion was syringeable and formed discrete particles when injected into the aqueous medium at 37° C.

### Example 49

Lutrol F-127 (a polyoxyethylene-polyoxypropylene block copolymer BASF), 1 g, and indomethacin 0.1 g, were dissolved in NMP, 2.3 g, to form the polymer phase. The novel gelled dispersion was further processed as per Example 1 with the oil phase comprising 2.5 g sorbitan monostearate (Arlacel 60), 0.2 g Tween 80 and 7.3 g sesame seed oil. The gelled dispersion was syringeable and formed discrete particles when injected into the aqueous medium at 37° C.

### Example 50

A gelled dispersion was prepared as described in Example 11, using Lutrol F-127 (a polyoxyethylene-polyoxypropylene block copolymer BASF), 30% w/w solution in a solvent phase comprising of 25:75% w/w DMA: PEG 400 and containing olanzapine, 10% w/w with respect to polymer, to form the polymer phase. The gelled dispersion was syringeable through a 18 gauge needle, formed discrete particles within 10 minutes upon coming in contact with the aqueous medium and was stable at room temperature for at least 8 hours.

### Example 51

A gelled dispersion was prepared as described in Example 11, using Lutrol F-68 (a polyoxyethylene-polyoxypropylene block copolymer BASF), 40% w/w solution in a solvent phase comprising of 25:75%w/w DMSO: PEG 400 and containing captopril, 10% w/w with respect to polymer, to form the polymer phase. The gelled dispersion was syringeable through a 18 gauge needle, formed discrete particles within 10 minutes upon coming in contact with the aqueous medium and was stable at room temperature for at least 22 days.

Those skilled in the art will recognize or be able to ascertain with simple routine experimentation, many equivalents of the specific embodiments of the invention described in the present specification. Such equivalents are intended to be encompassed in the scope of this specification.

1. A composition for providing an in-situ forming controlled release microcarrier delivery system, said composition being a gelled, syringeable droplet-in-oil dispersion comprising a biocompatible, biodegradable or non-biodegradable polymer in a water-soluble organic solvent and a pharmaceutically acceptable biocompatible emulsifier in solution in a biocompatible oil, wherein the biocompatible emulsifier comprises sorbitan monostearate, sorbitan monopalmitate or a mixture thereof, wherein the concentration of said polymer in solution in said solvent, and of said emulsifier in solution in said oil are effective to form an in-situ controlled release microcarrier delivery system when the dispersion comes into contact with an aqueous fluid.

2. The composition of claim 1, wherein said polymer is a biodegradable polymer selected from the group consisting essentially of polylactides, polyglycolides, polylactics, polylactic acid-co-glycolic acid, polylactide-co-glycolides, polyesteramides, star-branched polymers, polyphosphoesters, albumin, fibrin, fibrinogen combinations, polycaprolactones, polydioxanones, polycarbonates, polyhydroxybutyrates, polyalkylene oxalates, polyanhydrides, polyamides, polyurethanes, polyacetals, polyketals, polyorthocarbonates, polyphosphazenes, polyhydroxyvalerates, polyalkylene succinates, poly(malic acid), poly(amino acids), chitin, chitosan, polyorthoesters, gelatin, collagen, polyethylene glycols, polyethylene oxides, polypropylene oxides, polyethers, betacyclodextrin, polysaccharides, polyvinyl alcohol, polyvinyl pyrrolidone, polyvinyl-alcohol, polyoxyethylene-polypropylene block copolymers, and their copolymers, terpolymers and combinations and mixtures thereof.

3. The composition of claim 1, wherein said polymer is a nonbiodegradable polymer selected from the group consisting essentially of ethyl celluloses, acrylates, methacrylates, pyrrolidones, polyoxyethylenes, polyoxyethylene-polypropylene copolymers, hydroxypropylmethyl celluloses, hydroxypropyl celluloses, methyl celluloses, polymethylmethacrylates, cellulose acetates and their derivatives, shellac, methacrylic acid based polymers, their copolymers, combinations and mixtures thereof.

4. The composition of claim 1, wherein said solvent is selected from the group consisting essentially of N-methyl-2-pyrrolidone, NN′-dimethylacetamide, water, 2-pyrrolidone, sorbitol, dimethylsulfoxide, dimethylformamide, glycofural, glycerolformal, propylene glycol, polyethylene glycol, glycerol, caprolactam, decylmethyl sulfoxide, ethanol, dialkylamides, combinations and mixtures thereof.

5. The composition of claim 1, wherein said oil is selected from animal oils, isopropyl myristate, vegetable oils or their fractionated counterparts or their salts with other acids.

6. The composition of claim 1, wherein the sorbitan monostearate, sorbitan monopalmitate or a mixture thereof is capable of gelling the solvent and the oil.

7. The composition of claim 1, further comprising an amount of a biologically active agent selected from peptide drugs, protein drugs, desensitizing agents, antigens, vaccines, anti-infectives, antibiotics, antimicrobials, antineoplastics, antitumor, antiallergenics, steroidal anti-inflammatory agents, analgesics, decongestants, miotics, anticholinergics, sympathomimetics, sedatives, hypnotics, antipsychotics, psychic energizers, tranquilizers, androgenic steroids, estrogens, progestational agents, humoral agents, prostaglandins, analgesics, antispasmodics, antimalarials, antihistamines, cardioactive agents, non-steroidal anti-inflammatory agents, antiparkinsonian agents, antihypertensive agents, beta-adrenergic blocking agents, nutritional agents, antivirals, DNA fragments, nucleic acids, genetic material, oligonucleotides, radioisotopes, or combinations of these classes of compounds or other forms such as uncharged molecules, molecular complexes, salts, ethers, esters, amides, and other chemically modified forms of the biologically active agent which are biologically activated when injected into a body.

8. The composition of claim 1, further comprising a biologically active agent selected from leuprolide acetate, goserelin acetate, octreotide acetate, paclitaxel, chlorpheniramine maleate, trimethoprim, sulfamethoxazole, lactic acid, pseudoephedrine hydrochloride, olanzapine, captopril, lidocaine hydrochloride, felodipine, indomethacin, povidone iodine, or terbutaline sulfate.

9. The composition of claim 1, further comprising leuprolide acetate.

10. The composition of claim 1, further comprising paclitaxel.

11. The composition according to any one of claims 1-10, wherein the aqueous fluid is in a site within or on a body.

12. The composition according to claim 1, wherein the concentration of said polymer in said organic solvent in the polymer phase is between 1 and 90% w/w.

13. The composition according to claim 1, wherein the concentration of said emulsifier in respect to the polymer and organic solvent is between 5 and 50%w/w.

14. An in-situ formed controlled release microcarrier delivery system formed from the composition of claim 1, which system comprises microcarriers which are spherical, oblong, elliptical, or irregular in shape.

15. The system of claim 14, wherein the size of the microcarriers is between 1 to 400 μm.

16. The system of claim 14, wherein the size of the microcarriers is between 5 and 150 μm.

17. The system of claim 14, wherein greater than 40-60% of the microcarriers have a size of less than 100 μm.

18. A process for preparation of the composition of claim 1 which comprises the steps of:
(a) dissolving a biocompatible polymer or a mixture of polymers in a water-soluble organic solvent or a mixture of solvents at an elevated temperature to form a polymer solution,
(b) separately dissolving a biocompatible emulsifier in a biocompatible oil at an elevated temperature to form a continuous oil phase,
(c) emulsifying the polymer solution as described in (a) above into the continuous oil phase as described in (b) above to form a polymer droplet-in-oil dispersion, and
(d) mixing the polymer droplet-in-oil dispersion and subsequently cooling it to obtain a gelled dispersion.

19. The process of claim 18, wherein said polymer is a biodegradable polymer selected from the group consisting essentially of polylactides, polyglycolides, polylactics, polylactic acid-co-glycolic acid, polylactide-co-glycolides, polyesteramides, star-branched polymers, polyphosphoesters, albumin, fibrin, fibrinogen combinations, polycaprolactones, polydioxanones, polycarbonates, polyhydroxybutyrates, polyalkylene oxalates, polyanhydrides, polyamides, polyurethanes, polyacetals, polyketals, polyorthocarbonates, polyphosphazenes, polyhydroxyvalerates, polyalkylene succinates, poly(malic acid), poly(amino acids), chitin, chitosan, polyorthoesters, gelatin, collagen, polyethylene glycols, polyethylene oxides, polypropylene oxides, polyethers, betacyclodextrin, polysaccharides, polyvinyl alcohol, polyvinyl pyrrolidone, polyvinyl-alcohol, polyoxyethylene-polypropylene block copolymers, and their copolymers, terpolymers and combinations and mixtures thereof.

20. The process of claim 18, wherein said polymer is a nonbiodegradable polymer selected from the group consisting essentially of ethyl celluloses, acrylates, methacrylates, pyrrolidones, polyoxyethylenes, polyoxyethylene-polypropylene copolymers, hydroxypropylmethyl celluloses, hydroxypropyl celluloses, methyl celluloses, polymethylmethacrylates, cellulose acetates and their derivatives, shellac, methacrylic acid based polymers, their copolymers, combinations and mixtures thereof.

21. The process of claim 18, wherein said solvent is selected from the group consisting essentially of N-methyl-2-pyrrolidone, N,N′-dimethylacetamide, water, 2-pyrrolidone, sorbitol, dimethylsulfoxide, dimethylformamide, glycofural, glycerolformal, propylene glycol, polyethylene glycol, glycerol, caprolactam, decylmethyl sulfoxide, ethanol, dialkylamides, combinations and mixtures thereof.

22. The process of claim 18, wherein said oil is selected from animal oils, isopropyl myristate or vegetable oils or their fractionated counterparts or their salts with other acids.

23. The process of claim 18, wherein the sorbitan monostearate, sorbitan monopalmitate or a mixture thereof is capable of gelling the solvent and the oil phase.

24. The process of claim 18, further comprising a biologically active agent, a biologically inactive agent or both.

25. The process of claim 24, wherein the biologically active agent is selected from peptide drugs, protein drugs, desensitizing agents, antigens, vaccines, anti-infectives, antibiotics, antimicrobials, antineoplastics, antitumor, antiallergenics, steroidal anti-inflammatory agents, analgesics, decongestants, miotics, anticholinergics, sympathomimetics, sedatives, hypnotics, antipsychotics, psychic energizers, tranquilizers, androgenic steroids, estrogens, progestational agents, humoral agents, prostaglandins, analgesics, antispasmodics, antimalarials, antihistamines, cardioactive agents, non-steroidal anti-inflammatory agents, antiparkinsonian agents, antihypertensive agents, beta-adrenergic blocking agents, nutritional agents, antivirals, DNA fragments, nucleic acids, genetic material, oligonucleotides, radioisotopes, or combinations of these classes of compounds or other forms such as uncharged molecules, molecular complexes, salts, ethers, esters, amides, and other chemically modified forms of the biologically active agent which are biologically activated when injected into the body.

26. The process of claim 18, further comprising a biologically active agent which is selected from leuprolide acetate, goserelin acetate, octreotide acetate, paclitaxel, chlorpheniramine maleate, trimethoprim, sulfamethoxazole, lactic acid, pseudoephedrine hydrochloride, olanzapine, captopril, lidocaine hydrochloride, felodipine, indomethacin, povidone iodine, or terbutaline sulfate.

27. The process of claim 18, further comprising leuprolide acetate.

28. The process of claim 18, further comprising paclitaxel.

29. A kit for the in-situ formation of microcarriers which comprises:
a) a pharmaceutical composition for providing an in-situ forming controlled release microcarrier delivery system, said composition being a gelled, syringeable droplet-in-oil dispersion comprising a biocompatible, biodegradable or non-biodegradable polymer in a water-soluble organic solvent and a pharmaceutically acceptable biocompatible emulsifier in solution in a biocompatible oil, wherein the biocompatible emulsifier comprises sorbitan monostearate, sorbitan monopalmitate or mixture thereof wherein the concentration of said polymer in solution in said solvent, and of the emulsifier in solution in said oil are effective to form an in-situ controlled release microcarrier delivery system when said dispersion comes into contact with an aqueous fluid; and,
b) a device containing said pharmaceutical composition, said device having an inlet for the gelled dispersion, an ejector for expelling the gelled dispersion through an outlet into a site of a body.

30. The kit of claim 29, wherein said polymer is a biodegradable polymer selected from the group consisting essentially of polylactides, polyglycolides, polylactics, polylactic acid-co-glycolic acid, polylactide-co-glycolides, polyesteramides, star-branched polymers, polyphosphoesters, albumin, fibrin, fibrinogen combinations, polycaprolactones, polydioxanones, polycarbonates, polyhydroxybutyrates, polyalkylene oxalates, polyanhydrides, polyamides, polyurethanes, polyacetals, polyketals, polyorthocarbonates, polyphosphazenes, polyhydroxyvalerates, polyalkylene succinates, poly(malic acid), poly(amino acids), chitin, chitosan, polyorthoesters, gelatin, collagen, polyethylene glycols, polyethylene oxides, polypropylene oxides, polyethers, betacyclodextrin, polysaccharides, polyvinyl alcohol, polyvinyl pyrrolidone, polyvinyl-alcohol, polyoxyethylene-polypropylene block copolymers, and their copolymers, terpolymers and combinations and mixtures thereof.

31. The kit of claim 29, wherein said polymer is a nonbiodegradable polymer selected from the group consisting essentially of ethyl celluloses, acrylates, methacrylates, pyrrolidones, polyoxyethylenes, polyoxyethylene-polypropylene copolymers, hydroxypropylmethyl celluloses, hydroxypropyl celluloses, methyl celluloses, polymethylmethacrylates, cellulose acetates and their derivatives, shellac, methacrylic acid based polymers, their copolymers, combinations and mixtures thereof.

32. The kit of claim 29, wherein said solvent is selected from the group consisting essentially of N-methyl-2-pyrrolidone, N,N′-dimethylacetamide, water, 2-pyrrolidone, sorbitol, dimethylsulfoxide, dimethylformamide, glycofural, glycerolformal, propylene glycol, polyethylene glycol, glycerol, caprolactam, decylmethyl sulfoxide, ethanol, dialkylamides, combinations and mixtures thereof.

33. The kit of claim 29, wherein said oil is selected from animal oils, isopropyl myristate, vegetable oils or their fractionated counterparts or their salts with other acids.

34. The kit of claim 29, wherein the sorbitan monostearate, sorbitan monopalmitate or a mixture thereof is capable of gelling the solvent and the oil.

35. The kit of claim 29, further comprising a biologically active agent dissolved or dispersed within said gelled dispersion.

36. The kit of claim 29 further comprising a biologically active agent selected from peptide drugs, protein drugs, desensitizing agents, antigens, vaccines, anti-infectives, antibiotics, antimicrobials, antineoplastics, antitumor, antiallergenics, steroidal anti-inflammatory agents, analgesics, decongestants, miotics, anticholinergics, sympathomimetics, sedatives, hypnotics, antipsychotics, psychic energizers, tranquilizers, androgenic steroids, estrogens, progestational agents, humoral agents, prostaglandins, analgesics, antispasmodics, antimalarials, antihistamines, cardioactive agents, non-steroidal anti-inflammatory agents, antiparkinsonian agents, antihypertensive agents, beta-adrenergic blocking agents, nutritional agents, antivirals, DNA fragments, nucleic acids, genetic material, oligonucleotides, radioisotopes, or combinations of these classes of compounds or other forms such as uncharged molecules, molecular complexes, salts, ethers, esters, amides, and other chemically modified forms of the biologically active agent which are biologically activated when injected into the body.

37. The kit of claim 29, further comprising a biologically active agent selected from leuprolide acetate, goserelin acetate, octreotide acetate, paclitaxel, chlorpheniramine maleate, trimethoprim, sulfamethoxazole, lactic acid, pseudoephedrine hydrochloride, olanzapine, captopril, lidocaine hydrochloride, felodipine, indomethacin, povidone iodine, or terbutaline sulfate.

38. The kit of claim 29, further comprising leuprolide acetate.

39. The kit of claim 29, further comprising paclitaxel.

40. The kit according to claim 29, wherein the aqueous fluid is an aqueous body fluid.

41. A method of forming in-situ a controlled release microcarrier delivery system comprising:
(a) administering a pharmaceutical composition according to claim 1 to a site of a body and
(b) allowing the composition to come in contact with an aqueous fluid at the site of administration wherein an in-situ controlled release microcarrier delivery system is formed.

42. The method of claim 41, wherein said composition comprises a polymer which is a biodegradable polymer selected from the group consisting essentially of polylactides, polyglycolides, polylactics, polylactic acid-co-glycolic acid, polylactide-co-glycolides, polyesteramides, starbranched polymers, polyphosphoesters, albumin, fibrin, fibrinogen combinations, polycaprolactones, polydioxanones, polycarbonates, polyhydroxybutyrates, polyalkylene oxalates, polyanhydrides, polyamides, polyurethanes, polyacetals, polyketals, polyorthocarbonates, polyphosphazenes, polyhydroxyvalerates, polyalkylene succinates, poly(malic acid), poly(amino acids), chitin, chitosan, polyorthoesters, gelatin, collagen, polyethylene glycols, polyethylene oxides, polypropylene oxides, polyethers, betacyclodextrin, polysaccharides, polyvinyl alcohol, polyvinyl pyrrolidone, polyvinyl-alcohol, polyoxyethylene-polypropylene block copolymers, and their copolymers, terpolymers and combinations and mixtures thereof.

43. The method of claim 41, wherein said composition comprises a polymer which is is a non-biodegradable polymer selected from the group consisting essentially of ethyl celluloses, acrylates, methacrylates, pyrrolidones, polyoxyethylenes, polyoxyethylene-polypropylene copolymers, hydroxypropylmethyl celluloses, hydroxypropyl celluloses, methyl celluloses, polymethylmethacrylates, cellulose acetates and their derivatives, shellac, methacrylic acid based polymers, their copolymers, combinations and mixtures thereof.

44. The method of claim 41, wherein said composition comprises a solvent which is selected from the group consisting essentially of N-methyl-2-pyrrolidone, N,N′-dimethylacetamide, water, 2-pyrrolidone, sorbitol, dimethylsulfoxide, dimethylformamide, glycofural, glycerolformal, propylene glycol, polyethylene glycol, glycerol, caprolactam, decylmethyl sulfoxide, ethanol, dialkylamides, combinations and mixtures thereof.

45. The method of claim 41, wherein said wherein said oil is selected from animal oils, isopropyl myristate, vegetable oils or their fractionated counterparts or their salts with other acids.

46. The method of claim 41, wherein said composition further comprises a biologically active agent is selected from peptide drugs, protein drugs, desensitizing agents, antigens, vaccines, anti-infectives, antibiotics, antimicrobials, antineoplastics, antitumor, antiallergenics, steroidal anti-inflammatory agents, analgesics, decongestants, miotics, anticholinergics, sympathomimetics, sedatives, hypnotics, antipsychotics, psychic energizers, tranquilizers, androgenic steroids, estrogens, progestational agents, humoral agents, prostaglandins, analgesics, antispasmodics, antimalarials, antihistamines, cardioactive agents, non-steroidal anti-inflammatory agents, antiparkinsonian agents, antihypertensive agents, beta-adrenergic blocking agents, nutritional agents, antivirals, DNA fragments, nucleic acids, genetic material, oligonucleotides, radioisotopes, or combinations of these classes of compounds or other forms such as uncharged molecules, molecular complexes, salts, ethers, esters, amides, and other chemically modified forms of the biologically active agent which are biologically activated when injected into the body.

47. The method of claim 41, wherein the composition further comprises a biologically active agent which is selected from leuprolide acetate, goserelin acetate, octreotide acetate, paclitaxel, chlorpheniramine maleate, trimethoprim, sulfamethoxazole, lactic acid, pseudoephedrine hydrochloride, olanzapine, captopril, lidocaine hydrochloride, felodipine, indomethacin, povidone iodine, or terbutaline sulfate.

48. The method of claim 41, wherein composition further comprises leuprolide acetate.

49. The method of claim 47, wherein the composition further comprises paclitaxel.

50. The method of claim 41, wherein the body is an animal or human.

51. The method of claim 41, wherein the route of administration is selected from oral, buccal, ocular, nasal, rectal, vaginal, intravenous, intramuscular, subcutaneous, intraperitoneal, intradermal, intratumoral, intralesional, intravascular, topical, transdermal, local, regional, or loco-regional.

52. A method of preventing or treating a health disorder, disease or medical condition comprising administering a composition according to claim 1 to a patient in need thereof.

53. A method of preventing or treating a health disorder, disease or medical condition comprising using a kit according to claim 29 to form an in-situ controlled release microcarrier delivery system in a patient in need thereof.

54. The composition according to claim 5, wherein said animal oil is selected from whale oil or shark liver oil.

55. The composition according to claim 5, wherein the vegetable oil is selected from sesame seed oil, cottonseed oil, poppy seed oil, castor oil, coconut oil, canola oil, sunflower seed oil, peanut oil, corn oil, soyabean oil, or capric-caprylic triglycerides.

56. The composition according to claim 22, wherein said animal oil is selected from whale oil or shark liver oil.

57. The composition according to claim 22, wherein the vegetable oil is selected from sesame seed oil, cottonseed oil, poppy seed oil, castor oil, coconut oil, canola oil, sunflower seed oil, peanut oil, corn oil, soyabean oil, or capric-caprylic triglycerides.

58. The kit according to claim 33, wherein said animal oil is selected from whale oil or shark liver oil.

59. The kit according to claim 33, wherein the vegetable oil is selected from sesame seed oil, cottonseed oil, poppy seed oil, castor oil, coconut oil, canola oil, sunflower seed oil, peanut oil, corn oil, soyabean oil, or capric-caprylic triglycerides.

60. The method according to claim 45, wherein said animal oil is selected from whale oil or shark liver oil.

61. The method according to claim 45, wherein the vegetable oil is selected from sesame seed oil, cottonseed oil, poppy seed oil, castor oil, coconut oil, canola oil, sunflower seed oil, peanut oil, corn oil, soyabean oil, or capric-caprylic triglycerides.

62. The method according to claim 41 wherein the body is an aqueous medium.

63. The composition of claim 1 further comprising a biologically active agent, a biologically inactive agent or both.

64. The kit of claim 29 further comprising a biologically active agent, a biologically inactive agent or both.

65. The method of claim 41 further comprising a biologically active agent, a biologically inactive agent or both.

66. The process according to claim 18 further comprising adding a biologically active agent, bioinactive agent or both to the polymer solution formed in step (a).

67. The process according to claim 66 further comprising adding a biologically active agent, bioinactive agent or both to the continuous oil phase formed in step (b).

68. The process according to claim 18 further comprising adding a biologically active agent, bioinactive agent or both to the continuous oil phase formed in step (b).
